SEQUENCE LISTING (40 sequences)

SEQ ID NO: 1 - protein, 120 aa, Synthetic construct
QVQLVESGAEVKKPGSSVKVSCKSSGGTFNNYAVVWVRQAPGQGPEWMGGIIPMFDTPHYAQKFQGRVTITADEDTSTAYMELNSLRSDDTAVYYCATSYFRQWLPRDWGQGTLVTVSSA

SEQ ID NO: 2 - protein, 107 aa, Synthetic construct
EIVMTQSPSSLSASVGDRVTITCRASQGIRNELGWYQQKPGKAPRLLIYAASSLQSGVPSRFSGSGSGTDFTLTIGSLQPEDFATYYCLQDNNYPRTFGQGTKVDIK

SEQ ID NO: 3 - protein, 120 aa, Synthetic construct
EVQLVQSGAEVKKPGSSVKVSCLASGGTFNNYAVSWVRQAPGQGLEWMGGIIPMFDIAKYPQKFQGRVTITADESTSTAYMELSSLRSEDTAVYYCATSYFRQWLTWDWGQGTLVTVSSA

SEQ ID NO: 4 - protein, 109 aa, Synthetic construct
EIVLTQSPATLSLSPGQRATLSCGASQPVSSNYLAWFQHKPGLAPRLLIYDTSIRATGVPDRFSGSGSGTDFTLTISGLEPEDFAVYYCQQYGGSPQITFGGGTKVDIK

SEQ ID NO: 5 - protein, 120 aa, Synthetic construct
EVQLLESGAEVKKPGSSVKVSCKSSGGTFNNYAVVWVRQAPGQGPEWMGGIIPMFDTPHYAQKFQGRVTITADEDTSTAYMELNSLRSDDTAVYYCATSYFRQWLPRDWGQGTLVTVSSA

SEQ ID NO: 6 - protein, 109 aa, Synthetic construct
EIVLTQSPATLSLSPGQRATLSCGASQSVSSNYLAWYQQKPGLAPRLLIYDASIRATGIPDRFRGSGSGTDFTLTISGLEPEDFAVYYCHQYATAPQITFGGGTKVEIK

SEQ ID NO: 7 - protein, 122 aa, Synthetic construct
QVQLVESGGGLVQPGGSLRLSCAASGFTFSRYTMNWVRQAPGKGLEWVSYISSSSSIIHYADSVKGRFTISRDNAKKSLYLQMNSLRAEDTAVYYCASSEVVPATRYFDYWGQGTLVTVSSA

SEQ ID NO: 8 - protein, 120 aa, Synthetic construct
SYELTQPPSVSVAPGQTARITCGGNNIGTKSVHWYQQRPGRAPVLVVYDDSDRPSGIPERYSGSNSGNTATLTINRVEAGDEADYYCQVWDGISDHLEVFGGGTKLTVLGQPKAAPSVTL

SEQ ID NO: 9 - protein, 123 aa, Synthetic construct
EVQLVESGPGLVKPSETLSLTCTVSGGSISSYYWSWIRQPPGKGLEWIGYIYYSGSTNYNPSLKSRVSISVDTSKNQFSLRLSSVTAADTAVYYCAGHFRSSSSAKEYFHHWGQGTLVTVSSA

SEQ ID NO: 10 - protein, 107 aa, Synthetic construct
DIQLTQSPSSVSASVGDRVTITCRASQDISTWLAWYQQKPGKAPNLLIFAASSLQSGVPSRFSGDGSGTDFTLTISSLQPEDFATYYCQQASSFPYTFGQGTKVEIK

SEQ ID NO: 11 - protein, 8 aa, Synthetic construct
GGTFNNYA

SEQ ID NO: 12 - protein, 8 aa, Synthetic construct
IIPMFDTP

SEQ ID NO: 13 - protein, 12 aa, Synthetic construct
ATSYFRQWLPRD

SEQ ID NO: 14 - protein, 6 aa, Synthetic construct
QGIRNE

SEQ ID NO: 15 - skipped

SEQ ID NO: 16 - protein, 9 aa, Synthetic construct
LQDNNYPRT

SEQ ID NO: 17 - protein, 8 aa, Synthetic construct
GGTFNNYA

SEQ ID NO: 18 - protein, 8 aa, Synthetic construct
IIPMFDIA

SEQ ID NO: 19 - protein, 12 aa, Synthetic construct
ATSYFRQWLTWD

SEQ ID NO: 20 - protein, 7 aa, Synthetic construct
QPVSSNY

SEQ ID NO: 21 - skipped

SEQ ID NO: 22 - protein, 10 aa, Synthetic construct
QQYGGSPQIT

SEQ ID NO: 23 - protein, 8 aa, Synthetic construct
GGTFNNYA

SEQ ID NO: 24 - protein, 8 aa, Synthetic construct
IIPMFDTP

SEQ ID NO: 25 - protein, 12 aa, Synthetic construct
ATSYFRQWLPRD

SEQ ID NO: 26 - protein, 7 aa, Synthetic construct
QSVSSNY

SEQ ID NO: 27 - skipped

SEQ ID NO: 28 - protein, 10 aa, Synthetic construct
HQYATAPQIT

SEQ ID NO: 29 - protein, 8 aa, Synthetic construct
GFTFSRYT

SEQ ID NO: 30 - protein, 8 aa, Synthetic construct
ISSSSSII

SEQ ID NO: 31 - protein, 14 aa, Synthetic construct
ASSEVVPATRYFDY

SEQ ID NO: 32 - protein, 6 aa, Synthetic construct
NIGTKS

SEQ ID NO: 33 - skipped

SEQ ID NO: 34 - protein, 12 aa, Synthetic construct
QVWDGISDHLEV

SEQ ID NO: 35 - protein, 8 aa, Synthetic construct
GGSISSYY

SEQ ID NO: 36 - protein, 7 aa, Synthetic construct
IYYSGST

SEQ ID NO: 37 - protein, 16 aa, Synthetic construct
AGHFRSSSSAKEYFHH

SEQ ID NO: 38 - protein, 6 aa, Synthetic construct
QDISTW

SEQ ID NO: 39 - skipped

SEQ ID NO: 40 - protein, 9 aa, Synthetic construct
QQASSFPYT

(19) United States
(12) Patent Application Publication
(10) Pub. No.: US 2026/0317447 A1
(43) Pub. Date: Oct. 15, 2026

(54) HUMAN MONOCLONAL ANTIBODIES FOR TREATING SUBJECTS SUFFERING FROM PULMONARY FIBROSIS

(71) Applicant: The Johns Hopkins University, Baltimore, MD (US)

(72) Inventors: Felipe Andrade, Baltimore, MD (US); Erika L. Darrah, Baltimore, MD (US); Kristen DEMORUELLE, Baltimore, MD (US); Elizabeth REDENTE, Baltimore, MD (US)

(21) Appl. No.: 19/480,994

(22) PCT Filed: May 2, 2024
(86) PCT No.: PCT/US2024/027419

§ 371 Date: Nov. 3, 2025

Related U.S. Application Data
(60) Provisional application No. 63/464,242, filed on May 5, 2023.

(51) Int. Cl. C07K 16/40; A61K 39/00; A61P 11/00; A61P 19/02

(52) U.S. Cl. CPC C07K 16/40; A61P 11/00; A61P 19/02; A61K 2039/505

(57) ABSTRACT

Disclosed herein are anti-PAD4 antibodies and compositions comprising said monoclonal antibodies that can be used to treat pulmonary fibrosis in a subject in need of treatment thereof.

### RELATED APPLICATION INFORMATION

This application claims priority to U.S. Application No. 63/464,242, filed on May 5, 2023, the contents of which are herein incorporated by reference.

### SEQUENCE LISTING STATEMENT

The contents of the electronic sequence listing titled JHU_41732_601_SequenceListing.xml (Size: 33,397 bytes; and Date of Creation: May 2, 2024) is herein incorporated by reference in its entirety.

### FIELD

The present disclosure relates to anti-PAD4 monoclonal antibodies and compositions comprising said monoclonal antibodies that can be used to treat pulmonary fibrosis in a subject in need of treatment thereof.

### STATEMENT REGARDING FEDERALLY SPONSORED RESEARCH OR DEVELOPMENT

None.

### BACKGROUND

Defining the incidence rates of pulmonary fibrosis is challenging. One study has estimated the number of cases in the United States alone to be approximately 200,000, with an additional 50,000 cases diagnosed annually (1-5). This is a conservative estimate since this study focused on idiopathic pulmonary fibrosis, only a subset of lung fibrosis. Fibrotic lung disease can also occur in the setting of infection or autoimmune disease. Clinically significant interstitial lung disease (ILD) develops in 5-10% of patients with rheumatoid arthritis (RA) (18, 19), and most commonly occurs in a fibrotic pattern termed usual interstitial pneumonia (UIP). Patients with RA-ILD suffer impaired quality of life and activity-limiting dyspnea (20). RA-UIP is incurable and associated with markedly shortened survival (21). As such, RA-UIP has a major negative impact on tens of thousands of RA patients and improvements in the clinical management of RA-UIP are clearly needed. Therefore, there is an imperative need for new treatment strategies in RA-UIP and in fibrotic lung diseases more broadly.

Currently, there is one medication that has been approved for the treatment of RA-UIP (i.e. the tyrosine kinase inhibitor, nintedanib) (22), with its indication for use applying broadly in progressive pulmonary fibrosis and not specifically in RA-UIP. Nintedanib only modestly slows ILD progression, and the side effects make it difficult to use for many patients. Thus, there is a great need to develop more effective therapies that target disease-specific pathways in RA-UIP, which remain largely unknown.

### SUMMARY

The present disclosure relates to an isolated anti-PAD4 monoclonal antibody comprising:
a heavy variable chain comprising SEQ ID NO:1 and a light variable chain comprising SEQ ID NO:2;
a heavy variable chain comprising SEQ ID NO:3 and a light variable chain comprising SEQ ID NO:4;
a heavy variable chain comprising SEQ ID NO:5 and a light variable chain comprising SEQ ID NO:6;
a heavy variable chain comprising SEQ ID NO:7 and a light variable chain comprising SEQ ID NO:8; or
a heavy variable chain comprising SEQ ID NO:9 and a light variable chain comprising SEQ ID NO:10.

In some aspects, the above described monoclonal antibody is a human monoclonal antibody or a derivate or variant thereof. In another aspect the monoclonal antibody is a humanized antibody or a derivative or variant thereof. In still another aspect, the monoclonal antibody is a chimeric antibody or a derivative or variant thereof. In still yet another aspect, the monoclonal antibody is a bi-specific antibody or a derivative or variant thereof. In some aspects the monoclonal antibody is an IgG antibody.

In another aspect, the present disclosure relates to a composition comprising at least one of the above monoclonal antibodies.

In yet another aspect, the composition of the present disclosure further comprises an excipient or at least one pharmaceutically acceptable carrier.

In still a further aspect, the present disclosure relates to a kit comprising one or more of the above-described compositions.

In still yet a further aspect, the anti-PAD4 antibodies of the present disclosure can be used in methods of treating pulmonary fibrosis in a subject (e.g., such as a human). In some aspects, the method involves administering to the subject a therapeutically effective amount of any of the above compositions to treat the fibrosis. In some aspects, the subject is suffering from idiopathic lung fibrosis. In another aspect, the subject is suffering from interstitial lung disease. In yet further aspects, the subject is also suffering from rheumatoid arthritis.

Other aspects and embodiments of the disclosure will be apparent in light of the following detailed description and accompanying figures.

### DESCRIPTION OF THE FIGURES

FIG. 1 shows lung disease severity in anti-PAD4+ RA-UIP. Box plots characterizing the % predicted FVC (FIG. 1A), % predicted DLCO (FIG. 1B), and DTA fibrosis score (FIG. 1C) for subjects with RA-UIP when stratified by anti-PAD antibody positivity (anti-PAD4−, n=29; anti-PAD4+ mono-reactive, n=4; anti-PAD3/4XR+, n=4). Lower and upper box boundaries designate interquartile range (25th to 75th percentile). Line inside box designates median. Lower and upper error lines designate 5th and 95th percentile, respectively, and black circles designate points falling outside of this range. P-value based on Mann-Whitney test. FIG. 1D shows the survival curve in RA-UIP stratified by anti-PAD4 antibody positivity. Kaplan Meier estimates for % survival of all RA-UIP subjects stratified by anti-PAD4 positivity (red line—positive, blue line—negative).

FIG. 2 shows monocyte/macrophage polarization by anti-PAD4 mAbs. Healthy donor peripheral blood monocytes were (FIG. 2A) stimulated with anti-PAD4 clone 102 or isotype control antibodies for 24-hours (n=3) or (FIG. 2B) differentiated into non-polarized macrophages with M-CSF for 6 days (n=1). Macrophages were then incubated for 48 hours with M-CSF containing media alone, IFNγ+LPS, IL-4, anti-PAD4 mAb clone 102, or an isotype control antibody. Cytokine secretion was then measured by MSD multiplex array. Of note, the anti-PAD4 mAbs described herein (e.g., mAbs having a heavy variable chain comprising SEQ ID NO:7 and a light variable chain comprising SEQ ID NO:8) are purified in the mammalian Expi293 system and have endotoxin levels <1 EU/mg.

FIG. 3 shows the exogenous delivery of anti-PAD4 clone 17 mAbs (e.g., mAbs having a heavy variable chain comprising SEQ ID NO:5 and a light variable chain comprising SEQ ID NO:6) is protective against bleomycin-induced fibrosis development at day 21. FIG. 3A shows trichrome histology shows reduced fibrosis after anti-PAD4 Ab treatment compared to IgG2A control. FIG. 3B shows decreased lung collagen was observed and FIG. 3C shows BAL macrophages slightly decreased after anti-PAD4 Ab treatment. FIG. 3D shows TNF-α was significantly increased in the BALF of animals after treatment with anti-PAD4 Abs. FIG. 3E shows Hu-PAD4 was detectable by IP in serum of mice treated with anti-PAD4 Ab. n=3/group. ***p<0.001.

FIG. 4A shows the amino acid sequences of the monoclonal antibodies of the present disclosure. The CDRs in each of the variable heavy and light chains are indicated in underlining and bold font.

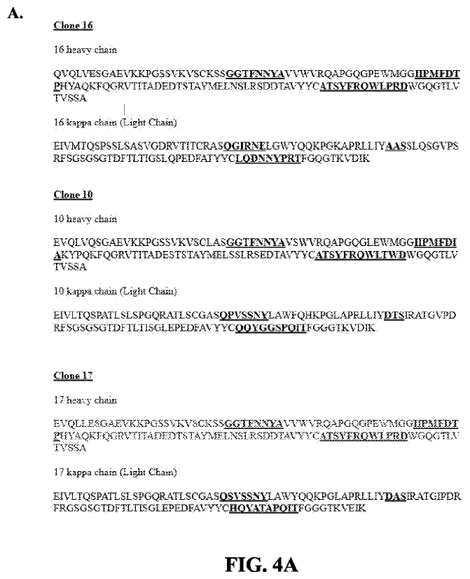

FIG. 4B shows the amino acid sequences of the monoclonal antibodies of the present disclosure. The CDRs in each of the variable heavy and light chains are indicated in underlining and bold font.

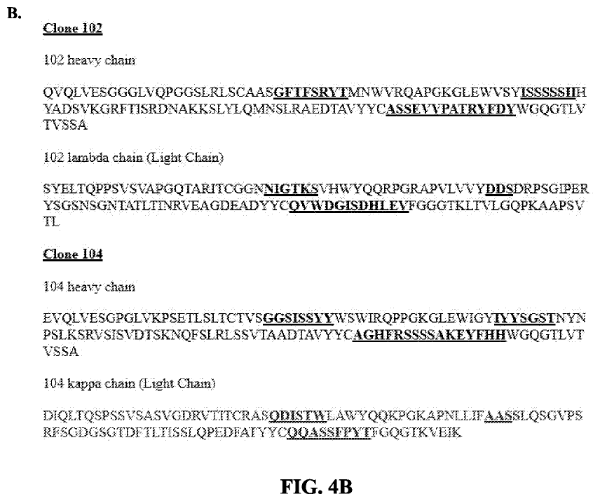

### DETAILED DESCRIPTION

Disclosed herein are compositions comprising monoclonal antibodies as well as compositions comprising said antibodies, methods of using said antibodies to screen for new treatments for autoimmune disease and kits containing said antibodies and compositions.

Section headings as used in this section and the entire disclosure herein are merely for organizational purposes and are not intended to be limiting.

### 1. Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

An “Affinity” refers to the strength of the sum total of noncovalent interactions between a single binding site of a molecule (e.g., a binding protein, such as, an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (KD). Affinity can be measured by common methods known in the art. For example, the affinity of an antibody for an antigen or epitope of interest can be measured using any art-recognized assay. Such methods include, for example, fluorescence activated cell sorting (FACS), separable beads (e.g., magnetic beads), antigen panning, and/or ELISA (see, e.g., Janeway et al. (eds.), Immunobiology, 5th ed., Garland Publishing, New York, N.Y., 2001).

An “Antibody” and “antibodies” as used herein refers to monoclonal antibodies, chimeric antibodies, multispecific antibodies, human antibodies, humanized antibodies (fully or partially humanized), animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F(ab′) fragments, F(ab′)₂ fragments, disulfide-linked Fvs (“sdFv”), and anti-idiotypic (“anti-Id”) antibodies, dual-domain antibodies, dual variable domain (DVD) or triple variable domain (TVD) antibodies (dual-variable domain immunoglobulins and methods for making them are described in Wu, C., et al., Nature Biotechnology, 25(11):1290-1297 (2007) and PCT International Application WO 2001/058956, the contents of each of which are herein incorporated by reference), and functionally active epitope-binding fragments of any of the above. Antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA, and IgY), class (for example, IgG1, IgG2, IgG3, IgG4, IgA1, and IgA2), or subclass.

A “Antibody fragment” as used herein refers to a portion of an intact antibody comprising the antigen-binding site or variable region. The portion does not include the constant heavy chain domains (i.e., CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)₂ fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

An “Autoimmune disease” as used herein refers to disorders associated with an undesired immune response in a subject. Autoimmune diseases fall in two broad categories: organ-specific and systemic. Autoimmune diseases include, without limitation, rheumatoid arthritis (RA), systemic lupus erythematosus (SLE), type I diabetes mellitus, type diabetes mellitus, multiple sclerosis (MS), neuromyelitis optica, immune-mediated infertility such as premature ovarian failure, scleroderma, Sjogren's disease, vitiligo, alopecia (baldness), polyglandular, Grave's disease, hypothyroidism, polymyositis, pemphigus vulgaris, pemphigus foliaceus, inflammatory bowel disease including Crohn's disease and ulcerative colitis, autoimmune hepatitis including that associated with hepatitis B virus (HBV) and hepatitis C virus (IICV), hypopituitarism, graft-versus-host disease (GvHD), myocarditis, Addison's disease, autoimmune skin diseases, uveitis, pernicious anemia, and hypoparathyroidism.

Autoimmune diseases may also include, without limitation, Hashimoto's thyroiditis, Type I and Type II autoimmune polyglandular syndromes, paraneoplastic pemphigus, bullus pemphigoid, dermatitis herpetiformis, linear IgA disease, epidermolysis bullosa acquisita, erythema nodosa, pemphigoid gestationis, cicatricial pemphigoid, mixed essential cryoglobulinemia, chronic bullous disease of childhood, hemolytic anemia, thrombocytopenic purpura, Goodpasture's syndrome, autoimmune neutropenia, myasthenia gravis, Eaton-Lambert myasthenic syndrome, stiff-man syndrome, acute disseminated encephalomyelitis, Guillain-Barre syndrome, chronic inflammatory demyelinating polyradiculoneuropathy, multifocal motor neuropathy with conduction block, chronic neuropathy with monoclonal gammopathy, opsonoclonus-myoclonus syndrome, cerebellar degeneration, encephalomyelitis, retinopathy, primary biliary sclerosis, sclerosing cholangitis, gluten-sensitive enteropathy, ankylosing spondylitis, reactive arthritides, polymyositis/dermatomyositis, mixed connective tissue disease, Bechet's syndrome, psoriasis, polyarteritis nodosa, allergic anguitis and granulomatosis (Churg-Strauss disease), polyangiitis overlap syndrome, hypersensitivity vasculitis, Wegener's granulomatosis, temporal arteritis, Takayasu's arteritis, Kawasaki's disease, isolated vasculitis of the central nervous system, thromboangiutis obliterans, sarcoidosis, glomerulonephritis, and cryopathies. These conditions are well known in the medical arts and are described, for example, in Harrison's Principles of Internal Medicine, 14^{th} ed., Fauci A S et al., eds., New York: McGraw-Hill, 1998.

A “Binding protein” is used herein to refer to a monomeric or multimeric protein that binds to and forms a complex with a binding partner, such as, for example, a polypeptide, an antigen, a chemical compound or other molecule, or a substrate of any kind. A binding protein specifically binds a binding partner. Binding proteins include antibodies, as well as antigen-binding fragments thereof and other various forms and derivatives thereof as are known in the art and described herein below, and other molecules comprising one or more antigen-binding domains that bind to an antigen molecule or a particular site (epitope) on the antigen molecule. Accordingly, a binding protein includes, but is not limited to, an antibody a tetrameric immunoglobulin, an IgG molecule, an IgG1 molecule, a monoclonal antibody, a chimeric antibody, a CDR-grafted antibody, a humanized antibody, a human antibody, an affinity matured antibody, and fragments of any such antibodies that retain the ability to bind to an antigen.

“Bispecific antibody” is used herein to refer to a full-length antibody that is generated by quadroma technology (see Milstein et al., Nature, 305(5934): 537-540 (1983)), by chemical conjugation of two different monoclonal antibodies (see, Staerz et al., Nature, 314(6012): 628-631 (1985)), or by knob-into-hole or similar approaches, which introduce mutations in the Fc region (see Holliger et al., Proc. Natl. Acad. Sci. USA, 90(14): 6444-6448 (1993)), resulting in multiple different immunoglobulin species of which only one is the functional bispecific antibody. A bispecific antibody binds one antigen (or epitope) on one of its two binding arms (one pair of HC/LC), and binds a different antigen (or epitope) on its second arm (a different pair of HC/LC). By this definition, a bispecific antibody has two distinct antigen-binding arms (in both specificity and CDR sequences) and is monovalent for each antigen to which it binds to. Bispecific antibodies can be made using the techniques as described in Brinkmann et al., MABS, 9(2):182-212 (2017), the contents of which are herein incorporated by reference.

A “CDR” is used herein to refer to the “complementarity determining region” within an antibody variable sequence. There are three CDRs in each of the variable regions of the heavy chain and the light chain. Proceeding from the N-terminus of a heavy or light chain, these regions are denoted “CDR1”, “CDR2”, and “CDR3”, for each of the variable regions. The term “CDR set” as used herein refers to a group of three CDRs that occur in a single variable region that binds the antigen. An antigen-binding site, therefore, may include six CDRs, comprising the CDR set from each of a heavy and a light chain variable region. A polypeptide comprising a single CDR, (e.g., a CDR1, CDR2, or CDR3) may be referred to as a “molecular recognition unit.” Crystallographic analyses of antigen-antibody complexes have demonstrated that the amino acid residues of CDRs form extensive contact with bound antigen, wherein the most extensive antigen contact is with the heavy chain CDR3. Thus, the molecular recognition units may be primarily responsible for the specificity of an antigen-binding site. In general, the CDR residues are directly and most substantially involved in influencing antigen binding.

The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as “Kabat CDRs”. Chothia and coworkers (Chothia and Lesk, J. Mol. Biol., 196: 901-917 (1987); and Chothia et al., Nature, 342: 877-883 (1989)) found that certain sub-portions within Kabat CDRs adopt nearly identical peptide backbone conformations, despite having great diversity at the level of amino acid sequence. These sub-portions were designated as “L1”, “L2”, and “L3”, or “H1”, “H2”, and “H3”, where the “L” and the “H” designate the light chain and the heavy chain regions, respectively. These regions may be referred to as “Chothia CDRs”, which have boundaries that overlap with Kabat CDRs. Other boundaries defining CDRs overlapping with the Kabat CDRs have been described by Padlan, FASEB J., 9: 133-139 (1995), and MacCallum, J. Mol. Biol., 262(5): 732-745 (1996). Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat- or Chothia-defined CDRs.

A “chimeric antibody” as used herein refers to a molecule in which different portions of the antibody are derived from different immunoglobulin molecules such as antibodies having a variable region derived from a non-human antibody and a human immunoglobulin constant region. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 6,311,415, 5,807,715, 4,816,567, and 4,816,397. Chimeric antibodies comprising one or more CDRs from a non-human species and framework regions from a human immunoglobulin molecule can be produced using a variety of techniques known in the art including, for example, CDR-grafting (EP 239,400; International Publication No. WO 91/09967; and U.S. Pat. Nos. 5,225,539, 5,530,101, and 5,585,089), veneering or resurfacing (EP 592,106; EP 519.596; Padlan, 1991, Molecular Immunology 28(4/5):489-498; Studnicka et al., 1994, Protein Engineering 7:805; and Roguska et al., 1994, Proc. Natl. Acad. Sci. USA 91:969), and chain shuffling (U.S. Pat. No. 5,565,332).

A “Derivative” as used herein refers to an antibody or antigen-binding fragment thereof that immunospecifically binds to an antigen, but which comprises, one, two, three, four, five or more amino acid substitutions, additions, deletions or modifications relative to a “parental” (or wild-type) molecule. Such amino acid substitutions or additions may introduce naturally occurring (i.e., DNA-encoded) or non-naturally occurring amino acid residues. In some aspects, “derivative” encompasses, for example, chimeric or humanized variants of any of antibodies 1.3, 4.5 or 7.8, as well as variants having altered CH1, hinge, CH2, CH3 or CH4 regions, so as to form, for example antibodies, etc., having variant Fe regions that exhibit enhanced or impaired effector or binding characteristics. In other aspects, “derivative” additionally encompasses non-amino acid modifications, for example, amino acids that may be glycosylated (e.g., have altered mannose, 2-N-acetylglucosamine, galactose, fucose, glucose, sialic acid, 5-N-acetylneuraminic acid, 5-glycolneuraminic acid, etc. content), acetylated, pegylated, phosphorylated, amidated, derivatized by known protecting/blocking groups, proteolytic cleavage, linked to a cellular ligand or other protein, etc. In some embodiments, the altered carbohydrate modifications modulate one or more of the following: solubilization of the antibody, facilitation of subcellular transport and secretion of the antibody, promotion of antibody assembly, conformational integrity, and antibody-mediated effector function. In a specific embodiment the altered carbohydrate modifications enhance antibody mediated effector function relative to the antibody lacking the carbohydrate modification. Carbohydrate modifications that lead to altered antibody mediated effector function are well known in the art (for example, see Shields, R. L. et al. (2002) “Lack Of Fucose On Hunan IgG N-Linked Oligosaccharide Improves Binding To Human Fcgamma RIII And Antibody-Dependent Cellular Toxicity,” J. Biol. Chem. 277(30): 26733-26740; Davies J. et al. (2001) “Expression Of GnTIII In A Recombinant Anti-CD20 CHO Production Cell Line: Expression Of Antibodies With Altered Glycoforms Leads To An Increase In ADCC Through Higher Affinity For FC Gamma RIII,” Biotechnology & Bioengineering 74(4): 288-294). Methods of altering carbohydrate contents are known to those skilled in the art, see, e.g., Wallick. S. C. et al. (1988) “Glycosylation Of A VH Residue Of A Monoclonal Antibody Against Alpha (1-6) Dextran Increases Its Affinity For Antigen,” J. Exp. Med. 168(3): 1099-109; Tao, M. H. et al. (1989) “Studies Of Aglycosylated Chimeric Mouse-Human IgG, Role Of Carbohydrate in The Structure And Effector Functions Mediated By The Human IgG Constant Region,” J. Immunol. 143(8): 2595-2601; Routledge, E. G. et al. (1995) “The Effect Of Aglycosylation On The Immunogenicity Of A Humanized Therapeutic CD3 Monoclonal Antibody,” Transplantation 60(8):847-53; Elliott. S. et al. (2003) “Enhancement Of Therapeutic Protein In Vivo Activities Through Glycoengineering,” Nature Biotechnol. 21:414-21; Shields. R. L. et al. (2002) “Lack of Fucose On Human IgG N-Linked Oligosaccharide Improves Binding To Human Fcgamma RIII And Antibody-Dependent Cellular Toxicity,” J. Biol. Chem. 277(30): 26733-26740).

A “Dual-specific antibody” is used herein to refer to a full-length antibody that can bind two different antigens (or epitopes) in each of its two binding arms (a pair of HC/LC) (see PCT publication WO 02/02773). Accordingly, a dual-specific binding protein has two identical antigen binding arms, with identical specificity and identical CDR sequences, and is bivalent for each antigen to which it binds.

A “Dual variable domain” is used herein to refer to two or more antigen binding sites on a binding protein, which may be divalent (two antigen binding sites), tetravalent (four antigen binding sites), or multivalent binding proteins. DVDs may be monospecific, i.e., capable of binding one antigen (or one specific epitope), or multispecific, i.e., capable of binding two or more antigens (i.e., two or more epitopes of the same target antigen molecule or two or more epitopes of different target antigens). A preferred DVD binding protein comprises two heavy chain DVD polypeptides and two light chain DVD polypeptides and is referred to as a “DVD immunoglobulin” or “DVD-Ig.” Such a DVD-Ig binding protein is thus tetrameric and reminiscent of an IgG molecule but provides more antigen binding sites than an IgG molecule. Thus, each half of a tetrameric DVD-Ig molecule is reminiscent of one half of an IgG molecule and comprises a heavy chain DVD polypeptide and a light chain DVD polypeptide, but unlike a pair of heavy and light chains of an IgG molecule that provides a single antigen binding domain, a pair of heavy and light chains of a DVD-Ig provide two or more antigen binding sites.

Each antigen binding site of a DVD-Ig binding protein may be derived from a donor (“parental”) monoclonal antibody and thus comprises a heavy chain variable domain (VH) and a light chain variable domain (VL) with a total of six CDRs involved in antigen binding per antigen binding site. Accordingly, a DVD-Ig binding protein that binds two different epitopes (i.e., two different epitopes of two different antigen molecules or two different epitopes of the same antigen molecule) comprises an antigen binding site derived from a first parental monoclonal antibody and an antigen binding site of a second parental monoclonal antibody.

A description of the design, expression, and characterization of DVD-Ig binding molecules is provided in PCT Publication No. WO 2007/024715, U.S. Pat. No. 7,612,181, and Wu et al., Nature Biotech., 25: 1290-1297 (2007). A preferred example of such DVD-Ig molecules comprises a heavy chain that comprises the structural formula VD1-(X1)n-VD2-C-(X2)n, wherein VD1 is a first heavy chain variable domain, VD2 is a second heavy chain variable domain, C is a heavy chain constant domain, X1 is a linker with the proviso that it is not CH1, X2 is an Fc region, and n is 0 or 1, but preferably 1; and a light chain that comprises the structural formula VD1-(X1)n-VD2-C-(X2)n, wherein VD1 is a first light chain variable domain, VD2 is a second light chain variable domain, C is a light chain constant domain, X1 is a linker with the proviso that it is not CH1, and X2 does not comprise an Fc region; and n is 0 or 1, but preferably 1. Such a DVD-Ig may comprise two such heavy chains and two such light chains, wherein each chain comprises variable domains linked in tandem without an intervening constant region between variable regions, wherein a heavy chain and a light chain associate to form tandem functional antigen binding sites, and a pair of heavy and light chains may associate with another pair of heavy and light chains to form a tetrameric binding protein with four functional antigen binding sites. In another example, a DVD-Ig molecule may comprise heavy and light chains that each comprise three variable domains (VD1, VD2, VD3) linked in tandem without an intervening constant region between variable domains, wherein a pair of heavy and light chains may associate to form three antigen binding sites, and wherein a pair of heavy and light chains may associate with another pair of heavy and light chains to form a tetrameric binding protein with six antigen binding sites.

In some aspects, a DVD-Ig binding protein not only binds the same target molecules bound by its parental monoclonal antibodies, but also possesses one or more desirable properties of one or more of its parental monoclonal antibodies. Preferably, such an additional property is an antibody parameter of one or more of the parental monoclonal antibodies. Antibody parameters that may be contributed to a DVD-Ig binding protein from one or more of its parental monoclonal antibodies include, but are not limited to, antigen specificity, antigen affinity, potency, biological function, epitope recognition, protein stability, protein solubility, production efficiency, immunogenicity, pharmacokinetics, bioavailability, tissue cross reactivity, and orthologous antigen binding.

A “Fragment antigen-binding fragment” or “Fab fragment” as used herein refers to a fragment of an antibody that binds to antigens and that contains one antigen-binding site, one complete light chain, and part of one heavy chain. A Fab is a monovalent fragment consisting of the VL, VH, CL and CH1 domains. A Fab is composed of one constant and one variable domain of each of the heavy and the light chain. The variable domain contains the paratope (the antigen-binding site), comprising a set of complementarity determining regions, at the amino terminal end of the monomer. Each arm of the Y thus binds an epitope on the antigen. Fab fragments can be generated such as has been described in the art, e.g., using the enzyme papain, which can be used to cleave an immunoglobulin monomer into two Fab fragments and an Fc fragment, or can be produced by recombinant means.

A “F(ab′)₂ fragment” as used herein refers to antibodies generated by pepsin digestion of whole IgG antibodies to remove most of the Fc region while leaving intact some of the hinge region. F(ab′)₂ fragments have two antigen-binding F(ab) portions linked together by disulfide bonds, and therefore are divalent with a molecular weight of about 110 kDa. Divalent antibody fragments (F(ab′)₂ fragments) are smaller than whole IgG molecules and enable a better penetration into tissue thus facilitating better antigen recognition in immunohistochemistry. The use of F(ab′)₂ fragments also avoids unspecific binding to Fc receptor on live cells or to Protein A/G. F(ab′)₂ fragments can both bind and precipitate antigens.

A “Framework” (FR) or “Framework sequence” as used herein may mean the remaining sequences of a variable region minus the CDRs. Because the exact definition of a CDR sequence can be determined by different systems (for example, see above), the meaning of a framework sequence is subject to correspondingly different interpretations. The six CDRs (CDR-L1, -L2, and -L3 of light chain and CDR-H1, -H2, and -H3 of heavy chain) also divide the framework regions on the light chain and the heavy chain into four sub-regions (FR1, FR2, FR3, and FR4) on each chain, in which CDR1 is positioned between FR1 and FR2, CDR2 between FR2 and FR3, and CDR3 between FR3 and FR4. Without specifying the particular sub-regions as FR1, FR2, FR3, or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region.

Human heavy chain and light chain FR sequences are known in the art that can be used as heavy chain and light chain “acceptor” framework sequences (or simply, “acceptor” sequences) to humanize a non-human antibody using techniques known in the art. In one embodiment, human heavy chain and light chain acceptor sequences are selected from the framework sequences listed in publicly available databases such as V-base or in the international ImMunoGeneTics® (IMGT®) information system.

A “Functional antigen binding site” as used herein may mean a site on a binding protein (e.g., an antibody) that is capable of binding a target antigen. The antigen binding affinity of the antigen binding site may not be as strong as the parent binding protein, e.g., parent antibody, from which the antigen binding site is derived, but the ability to bind antigen must be measurable using any one of a variety of methods known for evaluating protein, e.g., antibody, binding to an antigen. Moreover, the antigen binding affinity of each of the antigen binding sites of a multivalent protein, e.g., multivalent antibody, herein need not be quantitatively the same.

A “human antibody” as used herein refers to antibodies developed in a transgenic animal (e.g., mouse or rat) that have been genetically engineered with the human immunoglobulin locus (in contrast to humanized mAbs which are initially generated in wild type animal (e.g., mouse or rat) with a native genome bearing the mouse immunoglobulin locus). Human antibodies can be made by a variety of methods known in the art including phage display methods described above using antibody libraries derived from human immunoglobulin sequences (see U.S. Pat. Nos. 4,444,887 and 4,716,111; and International Publication Nos. WO 98/46645, WO 98/50433, WO 98/24893, WO 98/16654, WO 96/34096, WO 96/33735, and WO 91/10741). Human antibodies can be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stein cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized using conventional methodologies with a selected antigen, e.g., all or a portion of any of the polypeptides of antibodies A-E, described herein. Monoclonal antibodies directed against the antigen can be obtained from the immunized, transgenic mice using conventional hybridoma technology (see, e.g., U.S. Pat. No. 5,916,771). The human immunoglobulin transgenes harbored by the transgenic mice rearrange during B cell differentiation, and subsequently undergo class switching and somatic mutation. Thus, using such a technique, it is possible to produce therapeutically useful IgG. IgA, IgM and IgE antibodies. For an overview of this technology for producing human antibodies, see Lonberg and Huszar (1995, Int. Rev. Immunol. 13:65-93, which is incorporated herein by reference in its entirety). For a detailed discussion of this technology for producing human antibodies and human monoclonal antibodies and protocols for producing such antibodies, see, e.g., International Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. In addition, companies such as Abgenix, Inc. (Freemont. Calif.) and Medarex (Princeton, N.J.) can be engaged to provide human antibodies directed against a selected antigen using technology similar to that described above.

A “humanized antibody” as used herein refers to an immunoglobulin comprising a human framework region and one or more CDR's from a non-human (usually a transgenic animal such as a mouse or rat) immunoglobulin. The non-human immunoglobulin providing the CDR's is called the “donor” and the human immunoglobulin providing the framework is called the “acceptor.” Constant regions need not be present, but if they are, they must be substantially identical to human immunoglobulin constant regions. i.e., at least about 85-90%, preferably about 95% or more identical. Hence, all parts of a humanized immunoglobulin, except possibly the CDR's, are substantially identical to corresponding parts of natural human immunoglobulin sequences. A humanized antibody is an antibody comprising a humanized light chain and a humanized heavy chain immunoglobulin. For example, a humanized antibody would not encompass a typical chimeric antibody, because, e.g., the entire variable region of a chimeric antibody is non-human. One says that the donor antibody has been “humanized,” by the process of “humanization.” because the resultant humanized antibody is expected to bind to the same antigen as the donor antibody that provides the CDR's. For the most part, humanized antibodies are human immunoglobulins (recipient antibody) in which hypervariable region residues of the recipient are replaced by hypervariable region residues from a non-human species (donor antibody) such as mouse, rat, rabbit, or a non-human primate having the desired specificity, affinity, and capacity. In some instances, Framework Region (FR) residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence. The humanized antibody optionally also will comprise at least a portion of an immunoglobulin constant region (Fc) typically that of a human immunoglobulin that immunospecifically binds to an Fc RIIB polypeptide, that has been altered by the introduction of amino acid residue substitutions, deletions or additions (i.e., mutations). Humanized antibodies and methods for making such antibodies are described in, for example. European Patent Nos. EP 239,400, EP 592,106, and EP 519,596; International Publication Nos. WO 91/09967 and WO 93/17105; U.S. Pat. Nos. 5,225,539, 5,530,101, 5,565,332, 5,585,089, 5,766,886, and 6,407,213; and Padlan, 1991, Molecular Immunology 28(4/5):489-498; Studnicka et al., 1994, Protein Engineering 7(6):805-814; Roguska et al., 1994, PNAS 91:969-973; Tan et al., 2002, J. Immunol. 169:1119-1125; Caldas et al., 2000, Protein Eng. 13:353-360; Morea et al., 2000, Methods 20:267-79; Baca et al., 1997, J. Biol. Chem. 272:10678-10684; Roguska et al., 1996, Protein Eng. 9:895-904; Couto et al., 1995, Cancer Res. 55 (23 Supp):5973s-5977s; Couto et al., 1995, Cancer Res. 55:1717-22; Sandhu, 1994, Gene 150:409-10; Pedersen et al., 1994, J. Mol. Biol. 235:959-973; Jones et al., 1986, Nature 321:522-525; Reichmann et al., 1988, Nature 332:323-329; and Presta, 1992, Curr. Op. Struct. Biol. 2:593-596.

An “Identical” or “identity,” as used herein in the context of two or more polypeptide or polynucleotide sequences, can mean that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of the single sequence are included in the denominator but not the numerator of the calculation.

The terms “immunogen” and “antigen” are used interchangeably herein and refer to any molecule, compound, or substance that induces an immune response in an animal (e.g., a mammal). An “immune response” can entail, for example, antibody production and/or the activation of immune effector cells. An antigen in the context of the disclosure can comprise any subunit, fragment, or epitope of any proteinaceous or non-proteinaceous (e.g., carbohydrate or lipid) molecule that provokes an immune response in a mammal. By “epitope” is meant a sequence of an antigen that is recognized by a binding protein, an antibody, or an antigen receptor. Epitopes also are referred to in the art as “antigenic determinants.” In certain embodiments, an epitope is a region of an antigen that is specifically bound by an antibody. In certain embodiments, an epitope may include chemically active surface groupings of molecules such as amino acids, sugar side chains, phosphoryl, or sulfonyl groups. In certain embodiments, an epitope may have specific three-dimensional structural characteristics (e.g., a “conformational” epitope) and/or specific charge characteristics. The antigen can be a protein or peptide of viral, bacterial, parasitic, fungal, protozoan, prion, cellular, or extracellular origin, which provokes an immune response in a mammal, preferably leading to protective immunity.

An “Isolated polynucleotide” as used herein may mean a polynucleotide (e.g., of genomic, cDNA, or synthetic origin, or a combination thereof) that, by virtue of its origin, the isolated polynucleotide is not associated with all or a portion of a polynucleotide with which the “isolated polynucleotide” is found in nature; is operably linked to a polynucleotide that it is not linked to in nature; or does not occur in nature as part of a larger sequence.

A “Monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen (e.g., although cross-reactivity or shared reactivity may occur). Furthermore, in contrast to polyclonal antibody preparations that typically include different antibodies directed against different determinants (epitopes), each monoclonal antibody is directed against a single determinant on the antigen. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological. Methods for producing monoclonal antibodies are well known in the art. For example, monoclonal antibodies can be produced by: (1) fusion of sensitized lymphocytes and myelomas from different sources to produce continuous antibody-producing cell lines; (2) in vitro viral transformation of sensitized lymphocytes to form continuous antibody-producing cells; or (3) hybrid fusion of sensitized lymphocytes and continuous B lymphocyte cell lines (R. K Nakamura, “Monoclonal Antibodies: Methods and Clinical Lab Applications”, Clin. Physiol. Biochem, 1(2-5):160-72 (1983)).

A “Multivalent binding protein” is used herein to refer to a binding protein comprising two or more antigen binding sites (also referred to herein as “antigen binding domains”). A multivalent binding protein is preferably engineered to have three or more antigen binding sites and is generally not a naturally occurring antibody. The term “multispecific binding protein” refers to a binding protein that can bind two or more related or unrelated targets, including a binding protein capable of binding two or more different epitopes of the same target molecule.

A “peptide” or “polypeptide” is a linked sequence of two or more amino acids linked by peptide bonds. Peptides and polypeptides include proteins such as binding proteins, receptors, and antibodies. The terms “polypeptide” and “protein” are used interchangeably herein.

As used herein, the terms “providing,” “administering,” and “introducing” are used interchangeably herein and refer to the placement of the nanocomplexes and/or compositions of the present disclosure into a subject by a method or route which results in at least partial localization to a desired site.

A “Recombinant antibody” and “recombinant antibodies” refer to antibodies prepared by one or more steps, including cloning nucleic acid sequences encoding all or a part of one or more monoclonal antibodies into an appropriate expression vector by recombinant techniques and subsequently expressing the antibody in an appropriate host cell. The terms include, but are not limited to, recombinantly produced monoclonal antibodies, chimeric antibodies, humanized antibodies (fully or partially humanized), multi-specific or multi-valent structures formed from antibody fragments, bifunctional antibodies, heteroconjugate Abs, DVD-Ig®s, and other antibodies as described in herein (Dual-variable domain immunoglobulins and methods for making them are described in Wu, C., et al., Nature Biotechnology, 25:1290-1297 (2007)). The term “bifunctional antibody,” as used herein, refers to an antibody that comprises a first arm having a specificity for one antigenic site and a second arm having a specificity for a different antigenic site, i.e., the bifunctional antibodies have a dual specificity.

As used herein, when an antibody or other entity (e.g., antigen binding domain) “specifically recognizes” or “specifically binds” an antigen or epitope, it preferentially recognizes the antigen in a complex mixture of proteins and/or macromolecules, and binds the antigen or epitope with affinity which is substantially higher than to other entities not displaying the antigen or epitope. In this regard, “affinity which is substantially higher” means affinity that is high enough to enable detection of an antigen or epitope which is distinguished from entities using a desired assay or measurement apparatus. Typically, it means binding affinity having a binding constant (Kₐ) of at least 10⁷ M⁻¹ (e.g., >10⁷ M⁻¹, >10⁸ M⁻¹, >10⁹ M⁻¹, >10¹⁰ M⁻¹, >10¹¹ M⁻¹, >10¹² M⁻¹, >10¹³ M⁻¹, etc.). In certain such embodiments, an antibody is capable of binding different antigens so long as the different antigens comprise that particular epitope. In certain instances, for example, homologous proteins from different species may comprise the same epitope.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human, or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice, guinea pigs, and the like. Examples of non-mammals include, but are not limited to, birds, fish, and the like. In one embodiment, the mammal is a human.

As used herein, “treat,” “treating,” and the like means a slowing, stopping, or reversing of progression of a disease or disorder when provided a peptide or composition described herein to an appropriate subject. The term also includes a reversing of the progression of such a disease or disorder to a point of eliminating or greatly reducing the disease. As such, “treating” means an application or administration of the peptides or compositions described herein to a subject, where the subject has a disease or a symptom of a disease, where the purpose is to cure, heal, alleviate, relieve, alter, remedy, ameliorate, improve, or affect the disease or symptoms of the disease.

A “Variant” is used herein to describe a peptide or polypeptide that differs in amino acid sequence by the insertion, deletion, or conservative substitution of amino acids, but retain at least one biological activity. Representative examples of “biological activity” include the ability to be bound by a specific antibody or to promote an immune response. Variant is also used herein to describe a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree, and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### DETAILED DESCRIPTION

### Antibodies

In one aspect, the present disclosure relates to one or more anti-PAD4 monoclonal antibodies. In one aspect, the monoclonal antibodies are human monoclonal antibodies, humanized monoclonal antibodies, or chimeric antibodies having the following heavy and light chain variable sequences:

Clone 16 (Antibody A)-
CDRs are underlined and bolded
16 heavy chain
(SEQ ID NO: 1)
QVQLVESGAEVKKPGSSVKVSCKSSGGTENNYAVVWVRQAPGQGPEWMGG
IIPMFDTPHYAQKFQGRVTITADEDTSTAYMELNSLRSDDTAVYYCATSY
FRQWLPRDWGQGTLVTVSSA
16 kappa chain (light chain)
(SEQ ID NO: 2)
EIVMTQSPSSLSASVGDRVTITCRASQGIRNELGWYQQKPGKAPRLLIYA
ASSLQSGVPSRFSGSGSGTDFTLTIGSLOPEDFATYYCLQDNNYPRTFGQ
GTKVDIK
Clone 10 (Antibody B)-
CDRs are underlined and bolded
10 heavy chain
(SEQ ID NO: 3)
EVQLVQSGAEVKKPGSSVKVSCLASGGTFNNYAVSWVRQAPGQGLEWMGG
IIPMFDIAKYPQKFQGRVTITADESTSTAYMELSSLRSEDTAVYYCATSY
FRQWLTWDWGQGTLVTVSSA
10 kappa chain (light chain)
(SEQ ID NO: 4)
EIVLTQSPATLSLSPGQRATLSCGASQPVSSNYLAWFQHKPGLAPRLLIY
DTSIRATGVPDRFSGSGSGTDFTLTISGLEPEDFAVYYCQQYGGSPQITF
GGGTKVDIK
Clone 17 (Antibody C)-
CDRs are underlined and bolded
17 heavy chain
(SEQ ID NO: 5)
EVQLLESGAEVKKPGSSVKVSCKSSGGTFNNYAVVWVRQAPGQGPEWMGG
IIPMFDTPHYAQKFQGRVTITADEDTSTAYMELNSLRSDDTAVYYCATSY
FRQWLPRDWGQGTLVTVSSA
17 kappa chain (light chain)
(SEQ ID NO: 6)
EIVLTQSPATLSLSPGQRATLSCGASQSVSSNYLAWYQQKPGLAPRLLIY
DASIRATGIPDRFRGSGSGTDFTLTISGLEPEDFAVYYCHQYATAPQITF
GGGTKVEIK
Clone 102 (Antibody D)-
CDRs are underlined and bolded
102 heavy chain
(SEQ ID NO: 7)
QVQLVESGGGLVQPGGSLRLSCAASGFTFSRYTMNWVRQAPGKGLEWVSY
ISSSSSIIHYADSVKGRFTISRDNAKKSLYLQMNSLRAEDTAVYYCASSE
VVPATRYFDYWGQGTLVTVSSA
102 lambda chain (light chain)
(SEQ ID NO: 8)
SYELTQPPSVSVAPGQTARITCGGNNIGTKSVHWYQQRPGRAPVLVVYDD
SDRPSGIPERYSGSNSGNTATLTINRVEAGDEADYYCQVWDGISDHLEVF
GGGTKLTVLGQPKAAPSVTL
Clone 104 (Antibody E)-
CDRs are underlined and bolded
104 heavy chain
(SEQ ID NO: 9)
EVQLVESGPGLVKPSETLSLTCTVSGGSISSYYWSWIRQPPGKGLEWIGY
IYYSGSTNYNPSLKSRVSISVDTSKNQFSLRLSSVTAADTAVYYCAGHFR
SSSSAKEYFHHWGQGTLVTVSSA
104 kappa chain (light chain)
(SEQ ID NO: 10)
DIQLTQSPSSVSASVGDRVTITCRASQDISTWLAWYQQKPGKAPNLLIFA
ASSLQSGVPSRFSGDGSGTDFTLTISSLQPEDFATYYCQQASSFPYTFGQ
KGTVEIK.

In addition, all of the variable heavy and light chain sequences and their respective CDRs for clones 16, 10, 17, 102 and 104 are provided in the below Table 1:

TABLE 1

| | Variable Heavy/Variable | |
|---|---|---|
| Clone | Light Chain | CDRs |
| 16 | VH Chain: SEQ ID NO: 1 | Heavy Chain CDR1: GGTFNNYA (SEQ ID NO: 11) |
| 16 | VH Chain: SEQ ID NO: 1 | Heavy Chain CDR2: IIPMFDTP (SEQ ID NO: 12) |
| 16 | VH Chain: SEQ ID NO: 1 | Heavy Chain CDR3: ATSYFRQWLPRD (SEQ ID NO: 13) |
| 16 | VL Chain: SEQ ID NO: 2 | Light Chain CDR1: QGIRNE (SEQ ID NO: 14) |
| 16 | VL Chain: SEQ ID NO: 2 | Light Chain CDR2: AAS (SEQ ID NO: 15) |
| 16 | VL Chain: SEQ ID NO: 2 | Light Chain CDR3: LQDNNYPRT (SEQ ID NO: 16) |
| 10 | VH Chain: SEQ ID NO: 3 | Heavy Chain CDR1: GGTFNNYA (SEQ ID NO: 17) |
| 10 | VH Chain: SEQ ID NO: 3 | Heavy Chain CDR2: IIPMEDIA (SEQ ID NO: 18) |
| 10 | VH Chain: SEQ ID NO: 3 | Heavy Chain CDR3: ATSYFRQWLTWD (SEQ ID NO: 19) |
| 10 | VL Chain: SEQ ID NO: 4 | Light Chain CDR1: QPVSSNY (SEQ ID NO: 20) |
| 10 | VL Chain: SEQ ID NO: 4 | Light Chain CDR2: DTS (SEQ ID NO: 21) |
| 10 | VL Chain: SEQ ID NO: 4 | Light Chain CDR3: QQYGGSPQIT (SEQ ID NO: 22) |
| 17 | VH Chain: SEQ ID NO: 5 | Heavy Chain CDR1: GGTFNNYA (SEQ ID NO: 23) |
| 17 | VH Chain: SEQ ID NO: 5 | Heavy Chain CDR2: IIPMEDTP (SEQ ID NO: 24) |
| 17 | VH Chain: SEQ ID NO: 5 | Heavy Chain CDR3: ATSYFRQWLPRD (SEQ ID NO: 25) |
| 17 | VL Chain: SEQ ID NO: 6 | Light Chain CDR1: QSVSSNY (SEQ ID NO: 26) |
| 17 | VL Chain: SEQ ID NO: 6 | Light Chain CDR2: DAS(SEQ ID NO: 27) |
| 17 | VL Chain: SEQ ID NO: 6 | Light Chain CDR3: HQYATAPQIT (SEQ ID NO: 28) |
| 102 | VH Chain: SEQ ID NO: 7 | Heavy Chain CDR1: GFTFSRYT (SEQ ID NO: 29) |
| 102 | VH Chain: SEQ ID NO: 7 | Heavy Chain CDR2: ISSSSSII (SEQ ID NO: 30) |
| 102 | VH Chain: SEQ ID NO: 7 | Heavy Chain CDR3: ASSEVVPATRYFDY (SEQ ID NO: 31) |
| 102 | VL Chain: SEQ ID NO: 8 | Light Chain CDR1: NIGTKS (SEQ ID NO: 32) |
| 102 | VL Chain: SEQ ID NO: 8 | Light Chain CDR2: DDS (SEQ ID NO: 33) |
| 102 | VL Chain: SEQ ID NO: 8 | Light Chain CDR3: QVWDGISDHLEV (SEQ ID NO: 34) |
| 104 | VH Chain: SEQ ID NO: 9 | Heavy Chain CDR1: GGSISSYY (SEQ ID NO: 35) |
| 104 | VH Chain: SEQ ID NO: 9 | Heavy Chain CDR2: IYYSGST (SEQ ID NO: 36) |
| 104 | VH Chain: SEQ ID NO: 9 | Heavy Chain CDR3: AGHFRSSSSAKEYFHH (SEQ ID NO: 37) |
| 104 | VL Chain: SEQ ID NO: 10 | Light Chain CDR1: QDISTW (SEQ ID NO: 38) |
| 104 | VL Chain: SEQ ID NO: 10 | Light Chain CDR2: AAS (SEQ ID NO: 39) |
| 104 | VL Chain: SEQ ID NO: 10 | Light Chain CDR3: QQASSFPYT (SEQ ID NO: 40) |

In another aspect, the present disclosure relates to monoclonal antibodies comprising the below SEQ ID NOS. as well as any antigen binding fragments thereof comprising:
1) a heavy variable chain having at least 85%, at least 90%, at least 95%, at least 96%, at least 97%, at least 98%, at least 99% or at least 100% identity to SEQ ID NO:1 and a light variable chain having at least 85%, at least 90%, at least 95%, at least 96%, at least 97%, at least 98%, at least 99% or at least 100% identity to SEQ ID NO:2;
2) a heavy variable chain having at least 85%, at least 90%, at least 95%, at least 96%, at least 97%, at least 98%, at least 99% or at least 100% identity to SEQ ID NO:3 and a light variable chain having at least 85%, at least 90%, at least 95%, at least 96%, at least 97%, at least 98%, at least 99% or at least 100% identity SEQ ID NO:4;
3) a heavy variable chain having at least 85%, at least 90%, at least 95%, at least 96%, at least 97%, at least 98%, at least 99% or at least 100% identity to SEQ ID NO:5 and a light variable chain having at least 85%, at least 90%, at least 95%, at least 96%, at least 97%, at least 98%, at least 99% or at least 100% identity to SEQ ID NO:6;
4) a heavy variable chain having at least 85%, at least 90%, at least 95%, at least 96%, at least 97%, at least 98%, at least 99% or at least 100% identity to SEQ ID NO:7 and a light variable chain having at least 85%, at least 90%, at least 95%, at least 96%, at least 97%, at least 98%, at least 99% or at least 100% identity to SEQ ID NO:8; or
5) a heavy variable chain having at least 85%, at least 90%, at least 95%, at least 96%, at least 97%, at least 98%, at least 99% or at least 100% identity to SEQ ID NO:9 and a light variable chain having at least 85%, at least 90%, at least 95%, at least 96%, at least 97%, at least 98%, at least 99% or at least 100% identity to SEQ ID NO:10.

In another aspect, the present disclosure relates to monoclonal antibodies having the below SEQ ID NOS. as well as any antigen binding fragments thereof comprising:
1) a heavy variable chain comprising SEQ ID NO:1 and a light variable chain comprising SEQ ID NO:2;
2) a heavy chain CDR1 having the sequence of SEQ ID NO: 11, a heavy chain CDR2 having the sequence of SEQ ID NO:12; a heavy chain CDR3 having the sequence of SEQ ID NO:13; and
a light chain CDR1 having the sequence of SEQ ID NO: 14, a light chain CDR2 having the sequence of SEQ ID NO:15; a light chain CDR3 having the sequence of SEQ ID NO:16;
3) a heavy variable chain comprising SEQ ID NO:3 and a light variable chain comprising SEQ ID NO:4;
4) a heavy chain CDR1 having the sequence of SEQ ID NO: 17, a heavy chain CDR2 having the sequence of SEQ ID NO:18; a heavy chain CDR3 having the sequence of SEQ ID NO:19; and
a light chain CDR1 having the sequence of SEQ ID NO: 20, a light chain CDR2 having the sequence of SEQ ID NO:21; a light chain CDR3 having the sequence of SEQ ID NO:22;
5) a heavy variable chain comprising SEQ ID NO:5 and a light variable chain comprising SEQ ID NO:6;
6) a heavy chain CDR1 having the sequence of SEQ ID NO: 23, a heavy chain CDR2 having the sequence of SEQ ID NO:24; a heavy chain CDR3 having the sequence of SEQ ID NO:25; and
a light chain CDR1 having the sequence of SEQ ID NO: 26, a light chain CDR2 having the sequence of SEQ ID NO:27; a light chain CDR3 having the sequence of SEQ ID NO:28;
7) a heavy variable chain comprising SEQ ID NO:7 and a light variable chain comprising SEQ ID NO:8;
8) a heavy chain CDR1 having the sequence of SEQ ID NO: 29, a heavy chain CDR2 having the sequence of SEQ ID NO:30; a heavy chain CDR3 having the sequence of SEQ ID NO:31; and
a light chain CDR1 having the sequence of SEQ ID NO: 32, a light chain CDR2 having the sequence of SEQ ID NO:33; a light chain CDR3 having the sequence of SEQ ID NO:34;
9) a heavy variable chain comprising SEQ ID NO:9 and a light variable chain comprising SEQ ID NO:10; or
10) a heavy chain CDR1 having the sequence of SEQ ID NO: 35, a heavy chain CDR2 having the sequence of SEQ ID NO:36; a heavy chain CDR3 having the sequence of SEQ ID NO:37; and
a light chain CDR1 having the sequence of SEQ ID NO: 38, a light chain CDR2 having the sequence of SEQ ID NO:39; a light chain CDR3 having the sequence of SEQ ID NO:40.

In one aspect, of the monoclonal antibodies of the present disclosure are a human antibody, a humanized antibody, a chimeric antibody or any derivative or variant thereof. In other aspects, the monoclonal antibodies are bispecific antibodies or any derivative or variant thereof. In some embodiments, the antibody is a fragment selected from the group consisting of Fab, Fab-C, Fab′-SH, Fv, scFv, and (Fab′)2 fragments.

In another aspect, the monoclonal antibodies of the present disclosure are human IgG antibodies.

The antibodies of the present disclosure may be produced by any method known in the art useful for the production of polypeptides, e.g., in vitro synthesis, recombinant DNA production, and the like. In some aspects, any of antibodies A-E can be produced by recombinant DNA technology. Specifically, any of antibodies A-E may be produced using recombinant immunoglobulin expression technology. The recombinant production of immunoglobulin molecules, including humanized antibodies are described in U.S. Pat. Nos. 4,816,397, 6,331,415 and 4,816,567, U.K. patent GB 2,188,638, and U.K. patent GB 2,209,757. Techniques for the recombinant expression of immunoglobulins, including humanized immunoglobulins, can also be found, in Goeddel et al., Gene Expression Technology Methods in Enzymology Vol. 185 Academic Press (1991), and Borreback, Antibody Engineering, W. H. Freeman (1992). Additional information concerning the generation, design and expression of recombinant antibodies can be found in Mayforth, Designing Antibodies, Academic Press, San Diego (1993).

Recombinant chimeric antibodies of any of antibodies A-E can also be produced using techniques known in the art. For example, an exemplary process for the production of the recombinant chimeric antibodies for any of antibodies A-E can include the following: a) constructing, by conventional molecular biology methods, an expression vector that encodes and expresses an antibody heavy chain in which the CDRs and variable region of any of monoclonal antibodies A-E are fused to an Fc region derived from a human immunoglobulin, thereby producing a vector for the expression of a chimeric antibody heavy chain; b) constructing, by conventional molecular biology methods, an expression vector that encodes and expresses an antibody light chain of any of monoclonal antibodies A-E, thereby producing a vector for the expression of chimeric antibody light chain; c) transferring the expression vectors to a host cell by conventional molecular biology methods to produce a transfected host cell for the expression of chimeric antibodies; and d) culturing the transfected cell by conventional cell culture techniques so as to produce chimeric antibodies.

Recombinant humanized antibodies of any of antibodies A-E can also be produced using techniques known in the art. For example, an exemplary process for the production of recombinant humanized antibodies of any of antibodies A-E can include the following: a) constructing, by conventional molecular biology methods, an expression vector that encodes and expresses a heavy chain of any of antibodies A-E in which the CDRs and a minimal portion of the variable region framework that are required to retain donor antibody binding specificity are derived from a non-human immunoglobulin, such as any of monoclonal antibodies A-E, and the remainder of the antibody is derived from a human immunoglobulin, thereby producing a vector for the expression of a humanized antibody heavy chain; b) constructing, by conventional molecular biology methods, an expression vector that encodes and expresses an antibody light chain in which the CDRs and a minimal portion of the variable region framework that are required to retain donor antibody binding specificity are derived from a non-human immunoglobulin, such as any of monoclonal antibodies A-E, and the remainder of the antibody is derived from a human immunoglobulin, thereby producing a vector for the expression of humanized antibody light chain; c) transferring the expression vectors to a host cell by conventional molecular biology methods to produce a transfected host cell for the expression of humanized antibodies; and d) culturing the transfected cell by conventional cell culture techniques so as to produce humanized antibodies.

With respect to either of the above-described methods, host cells may be co-transfected with such expression vectors, which may contain different selectable markers but, with the exception of the heavy and light chain coding sequences, are preferably identical. This procedure provides for equal expression of heavy and light chain polypeptides. Alternatively, a single vector may be used which encodes both heavy and light chain polypeptides. The coding sequences for the heavy and light chains may comprise cDNA or genomic DNA or both. The host cell used to express any of the recombinant monoclonal antibodies A-E, can be either a bacterial cell such as Escherichia coli, or more preferably a eukaryotic cell (e.g., a Chinese hamster ovary (CHO) cell or a HEK-293 cell). The choice of expression vector is dependent upon the choice of host cell, and may be selected so as to have the desired expression and regulatory characteristics in the selected host cell. Other cell lines that may be used include, but are not limited to, CHO-K1, NSO, and PER.C6 (Crucell, Leiden, Netherlands).

Any of the above-described antibodies can be used to generate antiidiotype antibodies using techniques well known to those skilled in the art (see, e.g., Greenspan, N. S. et al. (1989) “Idiotypes: Structure and Immunogenicity,” FASEB J. 7:437-444; and Nisinoff, A. (1991) “Idiotypes: Concepts and Applications,” J. Immunol. 147(8):2429-2438).

The binding properties of any of the above antibodies can, if desired, be further improved by screening for variants that exhibit such desired characteristics. For example, such antibodies can be generated using various phage display methods known in the art. In phage display methods, functional antibody domains are displayed on the surface of phage particles which carry the polynucleotide sequences encoding them. In a particular embodiment, such phage can be utilized to display antigen binding domains, such as Fab and Fv or disulfide-bond stabilized Fv, expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage, including fd and M13. The antigen binding domains are expressed as a recombinantly fused protein to either the phage gene III or gene VIII protein. Examples of phage display methods that can be used to make the immunoglobulins, or fragments thereof, of the present disclosure include those disclosed in Brinkman, U. et al. (1995) “Phage Display Of Disulfide-Stabilized Fv Fragments,” J. Immunol. Methods, 182:41-50, 1995; Ames, R. S. et al. (1995) “Conversion Of Murine Fabs Isolated From A Combinatorial Phage Display Library To Full Length Immunoglobulins,” J. Immunol. Methods, 184:177-186; Kettleborough, C. A. et al. (1994) “Isolation Of Tumor Cell-Specific Single-Chain Fv From Immunized Mice Using Phage-Antibody Libraries And The Re-Construction Of Whole Antibodies From These Antibody Fragments,” Eur. J. Immunol., 24:952-958, 1994; Persic, L. et al. (1997) “An Integrated Vector System For The Eukaryotic Expression Of Antibodies Or Their Fragments After Selection From Phage Display Libraries,” Gene, 187:9-18; Burton, D. R. et al. (1994) “Human Antibodies From Combinatorial Libraries,” Adv. Immunol. 57:191-280; PCT Publications WO 92/001047; WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108.

As described in the above references, after phage selection, the antibody coding regions from the phage can be isolated and used to generate whole antibodies, including humanized antibodies, or any other desired fragments, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art (such as those disclosed in PCT Publication WO 92/22324; Mullinax, R. L. et al. (1992) “Expression Of A Heterodimeric Fab Antibody Protein In One Cloning Step,” BioTechniques, 12(6):864-869; and Sawai et al. (1995) “Direct Production Of The Fab Fragment Derived From The Sperm Immobilizing Antibody Using Polymerase Chain Reaction And cDNA Expression Vectors,” Am. J. Reprod. Immunol. 34:26-34; and Better, M. et al. (1988) “Escherichia coli Secretion Of An Active Chimeric Antibody Fragment,” Science 240:1041-1043). Examples of techniques which can be used to produce single-chain Fvs, and antibodies include those described in U.S. Pat. Nos. 4,946,778 and 5,258,498; Huston, J. S. et al. (1991) “Protein Engineering Of Single-Chain Fv Analogs And Fusion Proteins,” Methods in Enzymology 203:46-88; Shu, L. et al., “Secretion Of A Single-Gene-Encoded Immunoglobulin From Myeloma Cells,” Proc. Natl. Acad. Sci. (USA) 90:7995-7999; and Skerra. A. et al. (1988) “Assembly Of A Functional Immunoglobulin Fv Fragment In Escherichia coli,” Science 240:1038-1040.

Phage display technology can be used to increase the affinity of any of antibodies A-E. This technique would be useful in obtaining high affinity antibodies that could be used in the disclosed combinatorial methods. This technology, referred to as affinity maturation, employs mutagenesis or CDR walking and re-selection using such receptors or ligands (or their extracellular domains) or an antigenic fragment thereof to identify antibodies that bind with higher affinity to the antigen when compared with the initial or parental antibody (See, e.g., Glaser, S. M. et al. (1992) “Antibody Engineering By Codon-Based Mutagenesis In A Filamentous Phage Vector System,” J. Immunol. 149:3903-3913). Mutagenizing entire codons rather than single nucleotides results in a semi-randomized repertoire of amino acid mutations. Libraries can be constructed consisting of a pool of variant clones each of which differs by a single amino acid alteration in a single CDR, and which contain variants representing each possible amino acid substitution for each CDR residue. Mutants with increased binding affinity for the antigen can be screened by contacting the immobilized mutants with labeled antigen. Any screening method known in the art can be used to identify mutant antibodies with increased avidity to the antigen (e.g., ELISA) (see, e.g., Wu, H. et al. (1998) “Stepwise In Vitro Affinity Maturation Of Vitaxin, An Alphav Beta3-Specific Humanized Mab,” Proc. Natl. Acad. Sci. (USA) 95(11):6037-6042; Yelton, D. E. et al. (1995) “Affinity Maturation Of The BR96 Anti-Carcinoma Antibody By Codon-Based Mutagenesis,” J. Immunol. 155:1994-2004). CDR walking which randomizes the light chain may be used possible (see, Schier et al. (1996) “Isolation Of Picomolar Affinity Anti-C-Erbb-2 Single-Chain Fv By Molecular Evolution Of The Complementarity Determining Regions In The Center Of The Antibody Binding Site,” J. Mol. Biol. 263:551-567).

Also contemplated herein is the use of random mutagenesis to identify improved CDRs. Phage display technology can alternatively be used to increase (or decrease) CDR affinity. This technology, referred to as affinity maturation, employs mutagenesis or “CDR walking” and re-selection uses the target antigen or an antigenic fragment thereof to identify antibodies having CDRs that bind with higher (or lower) affinity to the antigen when compared with the initial or parental antibody (see, e.g., Glaser, S. M. et al. (1992) “Antibody Engineering By Codon-Based Mutagenesis In A Filamentous Phage Vector System,” J. Immunol. 149:3903-3913). Mutagenizing entire codons rather than single nucleotides results in a semi-randomized repertoire of amino acid mutations. Libraries can be constructed consisting of a pool of variant clones each of which differs by a single amino acid alteration in a single CDR and which contain variants representing each possible amino acid substitution for each CDR residue. Mutants with increased (or decreased) binding affinity for the antigen can be screened by contacting the immobilized mutants with labeled antigen. Any screening method known in the art can be used to identify mutant antibodies with increased (or decreased) avidity to the antigen (e.g., ELISA) (see, Wu, H. et al. (1998) “Stepwise In Vitro Affinity Maturation Of Vitaxin, An Alphav Beta3-Specific Humanized Mab,” Proc. Natl. Acad. Sci. (USA) 95(11):6037-6042; Yelton, D. E. et al. (1995) “Affinity Maturation Of The BR96 Anti-Carcinoma Antibody By Codon-Based Mutagenesis,” J. Immunol. 155:1994-2004). CDR walking which randomizes the light chain may be used possible (see, Schier et al. (1996) “Isolation Of Picomolar Affinity Anti-C-Erbb-2 Single-Chain Fv By Molecular Evolution Of The Complementarity Determining Regions In The Center Of The Antibody Binding Site,” J. Mol. Biol. 263:551-567).

Methods for accomplishing such affinity maturation are described for example in: Krause, J. C. et al. (2011) “An Insertion Mutation That Distorts Antibody Binding Site Architecture Enhances Function Of A Human Antibody,” MBio. 2(1) pii: e00345-10. doi: 10.1128/mBio.00345-10; Kuan, C. T. et al. (2010) “Affinity-Matured Anti-Glycoprotein NMB Recombinant Immunotoxins Targeting Malignant Gliomas And Melanomas,” Int. J. Cancer 10.1002/ijc.25645; Hackel, B. J. et al. (2010) “Stability And CDR Composition Biases Enrich Binder Functionality Landscapes,” J. Mol. Biol. 401(1):84-96; Montgomery, D. L. et al. (2009) “Affinity Maturation And Characterization Of A Human Monoclonal Antibody Against HIV-1 gp41,” MAbs 1(5):462-474; Gustchina, E. et al. (2009) “Affinity Maturation By Targeted Diversification Of The CDR-H2 Loop Of A Monoclonal Fab Derived From A Synthetic Naïve Human Antibody Library And Directed Against The Internal Trimeric Coiled-Coil Of Gp41 Yields A Set Of Fabs With Improved HIV-1 Neutralization Potency And Breadth,” Virology 393(1):112-119; Finlay, W. J. et al. (2009) “Affinity Maturation Of A Humanized Rat Antibody For Anti-RAGE Therapy: Comprehensive Mutagenesis Reveals A High Level Of Mutational Plasticity Both Inside And Outside The Complementarity-Determining Regions,” J. Mol. Biol. 388(3):541-558; Bostrom, J. et al. (2009) “Improving Antibody Binding Affinity And Specificity For Therapeutic Development,” Methods Mol. Biol. 525:353-376; Steidl, S. et al. (2008) “In Vitro Affinity Maturation Of Human GM-CSF Antibodies By Targeted CDR-Diversification,” Mol. Immunol. 46(1):135-144; and Barderas, R. et al. (2008) “Affinity maturation of antibodies assisted by in silico modeling,” Proc. Natl. Acad. Sci. (USA) 105(26):9029-9034.

The disclosure particularly contemplates the production and use of derivatives of any of the above-described antibodies and their antigen-binding fragments. In some embodiments, a humanized antibody is a derivative. Such a humanized antibody comprises amino acid residue substitutions, deletions, or additions in one or more non-human CDRs. The humanized antibody derivative may have substantially the same binding, better binding, or worse binding when compared to a non-derivative humanized antibody. In specific embodiments, one, two, three, four, or five amino acid residues of the CDR have been substituted, deleted, or added (i.e., mutated). A derivative antibody or antibody fragment may be modified by chemical modifications using techniques known to those of skill in the art, including, but not limited to, specific chemical cleavage, acetylation, formulation, metabolic synthesis of tunicamycin, etc. In one embodiment, an antibody derivative will possess a similar or identical function as the parental antibody. In another embodiment, an antibody derivative will exhibit an altered activity relative to the parental antibody. For example, a derivative antibody (or fragment thereof) can bind to its epitope more tightly or be more resistant to proteolysis than the parental antibody.

Derivatized antibodies may be used to alter the half-lives (e.g., serum half-lives) of parental antibodies in a mammal, preferably a human. Preferably such alteration will result in a half-life of greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the humanized antibodies of the present disclosure or fragments thereof in a mammal, preferably a human, results in a higher serum titer of said antibodies or antibody fragments in the mammal, and thus, reduces the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof with increased in vivo half-lives can be generated by modifying (e.g., substituting, deleting or adding) amino acid residues identified as involved in the interaction between the Fc domain and the FcRn receptor. The humanized antibodies of any of antibodies A-E can be engineered to increase biological half-lives (see, e.g. U.S. Pat. No. 6,277,375).

Antibodies or fragments thereof with increased in vivo half-lives can be generated by attaching to said antibodies or antibody fragments polymer molecules such as high molecular weight polyethyleneglycol (PEG). PEG can be attached to said antibodies or antibody fragments with or without a multifunctional linker either through site-specific conjugation of the PEG to the N- or C-terminus of said antibodies or antibody fragments or via epsilon-amino groups present on lysine residues. Linear or branched polymer derivatization that results in minimal loss of biological activity will be used. The degree of conjugation will be closely monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography. Alternatively, bispecific antibodies can be made using the techniques as described in Brinkmann et al., MABS, 9(2):182-212 (2017), the contents of which are herein incorporated by reference.

### Compositions

In another aspect, the present disclosure comprises a monoclonal antibody having:
1) a heavy variable chain comprising SEQ ID NO:1 and a light variable chain comprising SEQ ID NO:2;
2) a heavy chain CDR1 having the sequence of SEQ ID NO: 11, a heavy chain CDR2 having the sequence of SEQ ID NO:12; a heavy chain CDR3 having the sequence of SEQ ID NO:13; and
a light chain CDR1 having the sequence of SEQ ID NO: 14, a light chain CDR2 having the sequence of SEQ ID NO:15; a light chain CDR3 having the sequence of SEQ ID NO:16;
3) a heavy variable chain comprising SEQ ID NO:3 and a light variable chain comprising SEQ ID NO:4;
4) a heavy chain CDR1 having the sequence of SEQ ID NO: 17, a heavy chain CDR2 having the sequence of SEQ ID NO:18; a heavy chain CDR3 having the sequence of SEQ ID NO:19; and
a light chain CDR1 having the sequence of SEQ ID NO: 20, a light chain CDR2 having the sequence of SEQ ID NO:21; a light chain CDR3 having the sequence of SEQ ID NO:22;
5) a heavy variable chain comprising SEQ ID NO:5 and a light variable chain comprising SEQ ID NO:6;
6) a heavy chain CDR1 having the sequence of SEQ ID NO: 23, a heavy chain CDR2 having the sequence of SEQ ID NO:24; a heavy chain CDR3 having the sequence of SEQ ID NO:25; and
a light chain CDR1 having the sequence of SEQ ID NO: 26, a light chain CDR2 having the sequence of SEQ ID NO:27; a light chain CDR3 having the sequence of SEQ ID NO:28;
7) a heavy variable chain comprising SEQ ID NO:7 and a light variable chain comprising SEQ ID NO:8;
8) a heavy chain CDR1 having the sequence of SEQ ID NO: 29, a heavy chain CDR2 having the sequence of SEQ ID NO:30; a heavy chain CDR3 having the sequence of SEQ ID NO:31; and
a light chain CDR1 having the sequence of SEQ ID NO: 32, a light chain CDR2 having the sequence of SEQ ID NO:33; a light chain CDR3 having the sequence of SEQ ID NO:34;
9) a heavy variable chain comprising SEQ ID NO:9 and a light variable chain comprising SEQ ID NO: 10; or
10) a heavy chain CDR1 having the sequence of SEQ 11 NO: 35, a heavy chain CDR2 having the sequence of SEQ ID NO:36; a heavy chain CDR3 having the sequence of SEQ ID NO:37; and
a light chain CDR1 having the sequence of SEQ ID NO: 38, a light chain CDR2 having the sequence of SEQ ID NO:39; a light chain CDR3 having the sequence of SEQ ID NO:40.

In yet another aspect, the present disclosure comprises a monoclonal antibody having the below SEQ ID NOS. as well as any antigen binding fragments thereof comprising:
A) a heavy variable chain having at least 85%, at least 90%, at least 95%, at least 96%, at least 97%, at least 98%, at least 99% or at least 100% identity to SEQ ID NO:1 and a light variable chain having at least 85%, at least 90%, at least 95%, at least 96%, at least 97%, at least 98%, at least 99% or at least 100% identity to SEQ ID NO:2;
B) a heavy variable chain having at least 85%, at least 90%, at least 95%, at least 96%, at least 97%, at least 98%, at least 99% or at least 100% identity to SEQ ID NO:3 and a light variable chain having at least 85%, at least 90%, at least 95%, at least 96%, at least 97%, at least 98%, at least 99% or at least 100% identity SEQ ID NO:4;
C) a heavy variable chain having at least 85%, at least 90%, at least 95%, at least 96%, at least 97%, at least 98%, at least 99% or at least 100% identity to SEQ ID NO:5 and a light variable chain having at least 85%, at least 90%, at least 95%, at least 96%, at least 97%, at least 98%, at least 99% or at least 100% identity to SEQ ID NO:6;
D) a heavy variable chain having at least 85%, at least 90%, at least 95%, at least 96%, at least 97%, at least 98%, at least 99% or at least 100% identity to SEQ ID NO:7 and a light variable chain having at least 85%, at least 90%, at least 95%, at least 96%, at least 97%, at least 98%, at least 99% or at least 100% identity to SEQ ID NO:8; or
E) a heavy variable chain having at least 85%, at least 90%, at least 95%, at least 96%, at least 97%, at least 98%, at least 99% or at least 100% identity to SEQ ID NO:9 and a light variable chain having at least 85%, at least 90%, at least 95%, at least 96%, at least 97%, at least 98%, at least 99% or at least 100% identity to SEQ ID NO:10 and at least one excipient or pharmaceutically acceptable carrier.

The choice of pharmaceutically acceptable excipient or pharmaceutically acceptable carrier will depend on factors including, but not limited to, the particular mode of administration, the effect of the excipient on solubility and stability, and the nature of the dosage form. The compositions of the present invention will be readily apparent to those skilled in the art. Techniques and formulations may be found, for example, in Remington's Pharmaceutical Sciences, 19th Edition (Mack Publishing Company, 1995).

The term “pharmaceutically acceptable carrier,” as used herein, means a non-toxic, inert solid, semi-solid or liquid filler, diluent, encapsulating material, surfactant, cyclodextrins or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; surfactants such as, but not limited to, cremophor EL, cremophor RH 60, Solutol HS 15 and polysorbate 80; cyclodextrins such as, but not limited to, alpha-CD, beta-CD, gamma-CD, HP-beta-CD, SBE-beta-CD; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as releasing agents, coating agents, preservatives and antioxidants can also be present in the composition, according to the judgment of the formulator.

The route by which the disclosed antibodies are administered, and the form of the composition will dictate the type of carrier to be used. The composition may be in a variety of forms, suitable, for example, for systemic administration (e.g., oral, rectal, nasal, sublingual, buccal, implants, or parenteral injections) or topical administration (e.g., dermal, pulmonary, nasal, aural, ocular, liposome delivery systems, or iontophoresis). In some aspects, the antibodies or compositions are administered to a patient by inhalation.

### Methods of Treatment

Also disclosed herein are methods of using the anti-PAD4 antibodies described herein to treat pulmonary fibrosis in a subject in need of treatment thereof. In some aspects, the method comprises administering to the subject in need of treatment a therapeutically effective amount of one or more of the above-described compositions. In some aspects, the subject to be treated is suffering from idiopathic lung fibrosis. In another aspect, the subject to be treated is suffering from interstitial lung disease. In still another aspect, the subject is suffering from rheumatoid arthritis. The amount of the anti-PAD4 antibodies described herein that can be administered to a subject to treat the pulmonary fibrosis, such as a human, can be determined by a clinician using routine techniques known in the art. For example, the amount of any of anti-PAD4 antibodies described herein can be administered to a subject to treat pulmonary fibrosis is from about 1.0 mg/kg to about 1000 mg/kg.

While not wishing to be bound by any theory, it is believed that the anti-PAD4 monoclonal antibodies described herein preferentially bind to monocytes, and stimulate secretion of TNF-α by human monocytes and monocyte-derived macrophages in vitro. This is significant because the pathogenesis of fibrotic lung diseases generally involves activation of lung fibroblasts that transform into pro-fibrotic myofibroblasts, in a process largely driven by TGF-β resulting in the accumulation of apoptotic-resistant pro-fibrotic lung myofibroblasts. Importantly, TNF-α sensitizes myofibroblasts to FAS ligand mediated apoptosis and exogenous delivery of TNF-α accelerates fibrosis resolution in vivo. This mechanism is supported by the data in the example which demonstrates that higher levels of sputum TNF-α in rheumatoid arthritis patients with anti-PAD4 antibodies and increased survival in anti-PAD4 positive RA patients with fibrotic lung disease compared to anti-PAD4 negative individuals.

### Kits

Also disclosed herein are kits comprising one or more of the antibodies (e.g., compositions) as disclosed herein.

The kits can also comprise other agents (e.g., pharmaceutical agents) and/or products co-packaged, co-formulated, and/or co-delivered with other components. The kits can also comprise instructions for using the components of the kit. The instructions are relevant materials or methodologies pertaining to the kit. The materials may include any combination of the following: background information, list of components, brief or detailed protocols for using the monoclonal antibodies described herein, troubleshooting, references, technical support, and any other related documents. Instructions can be supplied with the kit or as a separate member component, either as a paper form or an electronic form which may be supplied on computer readable memory device or downloaded from an internet website, or as recorded presentation.

It is understood that the disclosed kits can be employed in connection with the disclosed methods. The kit may further contain containers or devices for use with the methods disclosed herein. For example, the kits can comprise delivery devices (e.g., syringes).

The kits provided herein are in suitable packaging. Suitable packaging includes, but is not limited to, vials, bottles, jars, flexible packaging, and the like. Individual member components of the kits may be physically packaged together or separately.

The present disclosure has multiple aspects, illustrated by the following non-limiting examples.

### EXAMPLES

### Methods

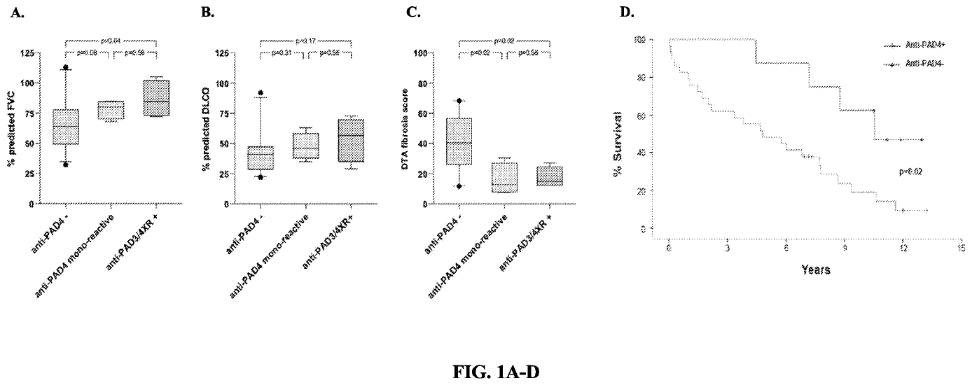

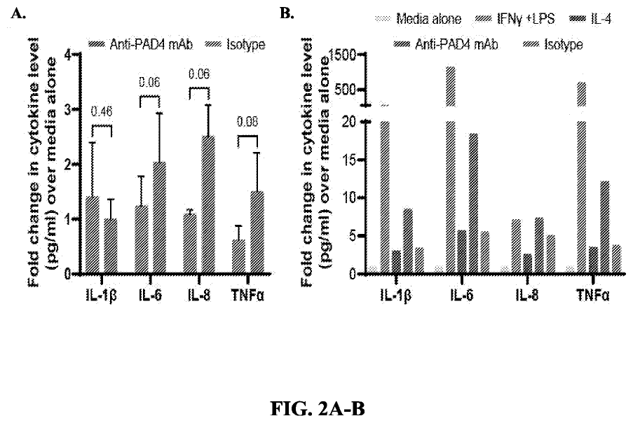

Anti-PAD4 Abs in RA and RA-UIP. Anti-PAD4-IgG Abs are present in the serum of 23-45% of all RA patients (35-37). It was found (See, Wilson et al., Am. J. Med. Sci., pages 1-8 (2023), the contents of which are herein incorporated by reference) that serum anti-PAD4-IgG Abs were present in 22% (8/37) of RA patients with clinically significant RA-UIP and were highly specific for RA-UIP compared to healthy controls (0%, n=25) and patients with IPF (0%, n=32). Importantly, it was found that serum anti-PAD4 Abs were associated with less lung fibrosis (by qCT), better lung function (by FVC) and decreased mortality in RA-UIP (FIG. 1 and FIG. 2). Of note, serum anti-PAD4-IgG Abs are also present in RA patients without ILD (35-37), suggesting that these Abs are not generated in response to RA-UIP but instead likely modulate RA-UIP pathogenesis when present.

Monocyte/macrophage activation by anti-PAD4 Abs and role of TNF-α in pulmonary fibrosis. To define whether human anti-PAD4 mAbs could exert direct cellular effects, primary human monocytes were cultured from three healthy donors with human anti-PAD4 clone 102 (SEQ ID NOS: 7 and 8) or an isotype control. Increased IL-6, IL-8, and TNF-α production by monocytes were observed in response to anti-PAD4 mAb, but no change in IL-1β levels (FIG. 2A). Similarly, we observed an increase in pro-inflammatory cytokine secretion by non-polarized human macrophages in response to human anti-PAD4 mAb, that was not observed with the isotype control (FIG. 2B). These data support that anti-PAD4 Abs can have direct cellular effects on human monocytes and macrophages resulting in a pro-inflammatory phenotype with increased TNF-α production.

These in vitro findings also parallel increased sputum levels of TNF-α that were found ex vivo when comparing serum anti-PAD4 Ab positive vs. anti-PAD4 Ab negative RA subjects (mean (SD); 30.5 (25) pg/mL vs. 12.7 (7) pg/mL, respectively). These preliminary data were generated in an RA convenience cohort that did not have ILD. However, these data support a relationship between anti-PAD4 Abs and a lung phenotype that could antagonize pro-fibrotic pathways in human subjects with RA. Of note, TNF-α receptor signaling has been associated with lung fibroblast apoptosis in cell culture and decreased lung fibrosis in animal models (12, 14, 43, 44). In fact, fibrosis is prolonged in TNF-α^{-/-} mice and resolves with lung delivery of TNF-α due in part to the activation of pro-inflammatory macrophages (14).

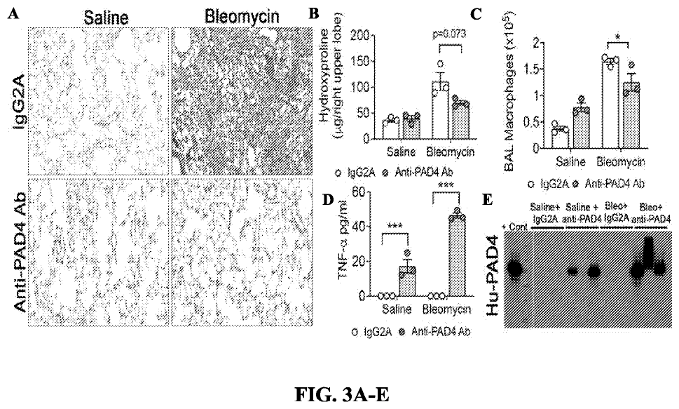

Anti-PAD4 Abs abrogate lung fibrosis in vivo. The in vivo effects of recombinant human anti-PAD4 clone 17 mAbs (namely, mAbs having a heavy variable chain comprising SEQ ID NO:5 and a light variable chain comprising SEQ ID NO:6) were defined using the bleomycin model of lung fibrosis, in which murine-compatible anti-PAD4 mAbs were intratracheally administered, in which the human heavy and light chain constant regions were replaced with murine IgG2a and kappa chains respectively, and IgG2a control at day 8, 12 and 15 after bleomycin administration. Lung fibrosis was evaluated at day 21 to assess prevention of fibrosis development with anti-PAD4 mAb administration. Compared to mice who received IgG control, mice who received anti-PAD4 mAbs had a reduction in lung fibrosis, as measured by histology and lung collagen levels measured by hydroxyproline (FIG. 3A and FIG. 3B). Lung macrophage counts were decreased in mice who received anti-PAD4 mAbs, as would be expected given the reduced level of lung fibrosis (FIG. 3C). Importantly, BALF TNF-α levels were increased in mice who received anti-PAD4 mAbs (FIG. 3D), supporting the hypothesis that anti-PAD4 Abs are modulating macrophage function.

Together, this data suggest that anti-PAD4 Abs, namely, those mAbs having A) a heavy variable chain comprising SEQ ID NO:5 and a light variable chain comprising SEQ ID NO:6; B) a heavy variable chain comprising SEQ ID NO:7 and a light variable chain comprising SEQ ID NO:8, attenuate lung fibrosis by modulating lung monocyte/macrophage production of the pro-inflammatory cytokine TNF-α.

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the non-limiting examples described herein.

It is understood that the foregoing detailed description and accompanying examples are merely illustrative and are not to be taken as limitations upon the scope of the present disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the present disclosure, may be made without departing from the spirit and scope thereof.

### REFERENCES

1. Agostini C, Gurrieri C. Chemokine/cytokine cocktail in idiopathic pulmonary fibrosis. Proc Am Thorac Soc 2006; 3: 357-363.
2. Phan S H. Genesis of the myofibroblast in lung injury and fibrosis. Proc Am Thorac Soc 2012; 9: 148-152.
3. Wuyts W A, Agostini C, Antoniou K M, Bouros D, Chambers R C, Cottin V, Egan J J, Lambrecht B N, Lories R, Parfrey H, Prasse A, Robalo-Cordeiro C. Verbeken E, Verschakelen J A, Wells A U, Verleden G M. The pathogenesis of pulmonary fibrosis: a moving target. Eur Respir J 2013; 41: 1207-1218.
4. Fernandez I E, Eickelberg O. The impact of TGF-beta on lung fibrosis: from targeting to biomarkers. Proc Am Thorac Soc 2012; 9: 111-116.
5. Laurent G J, McAnulty R J, Hill M, Chambers R. Escape from the matrix: multiple mechanisms for fibroblast activation in pulmonary fibrosis. Proc Am Thorac Soc 2008; 5: 311-315.
6. Gregory A D, Kliment C R, Metz H E, Kim K H, Kargl J, Agostini B A, Crum L T, Oczypok E A, Oury T A, Houghton A M. Neutrophil elastase promotes myofibroblast differentiation in lung fibrosis. J Leukoc Biol 2015; 98: 143-152.
7. Byrne A J, Maher T M, Lloyd C M. Pulmonary Macrophages: A New Therapeutic Pathway in Fibrosing Lung Disease? Trends Mol Med 2016; 22: 303-316.
8. Takemasa A, Ishii Y, Fukuda T. A neutrophil elastase inhibitor prevents bleomycin-induced pulmonary fibrosis in mice. Eur Respir J 2012; 40: 1475-1482.
9. Mitsuhashi H, Asano S, Nonaka T, Hamamura I, Masuda K, Kiyoki M. Administration of truncated secretory leukoprotease inhibitor ameliorates bleomycin-induced pulmonary fibrosis in hamsters. Am J Respir Crit Care Med 1996; 153: 369-374.
10. Gibbons M A, MacKinnon A C, Ramachandran P, Dhaliwal K, Duffin R, Phythian-Adams A T, van Rooijen N, Haslett C, Howie S E, Simpson A J, Hirani N, Gauldie J, Iredale J P, Sethi T, Forbes S J. Ly6Chi monocytes direct alternatively activated profibrotic macrophage regulation of lung fibrosis. Am J Respir Crit Care Med 2011; 184: 569-581.
11. Wynn T A, Vannella K M. Macrophages in Tissue Repair, Regeneration, and Fibrosis. Immunity 2016; 44: 450-462.
12. Wynes M W, Edelman B L, Kostyk A G, Edwards M G, Coldren C, Groshong S D, Cosgrove G P, Redente E F, Bamberg A, Brown K K, Reisdorph N, Keith R C, Frankel S K, Riches D W. Increased cell surface Fas expression is necessary and sufficient to sensitize lung fibroblasts to Fas ligation-induced apoptosis: implications for fibroblast accumulation in idiopathic pulmonary fibrosis. J Immunol 2011; 187: 527-537.
13. Bamberg A, Redente E F, Groshong S D, Tuder R M, Cool C D, Keith R C, Edelman B L, Black B P, Cosgrove G P, Wynes M W, Curran-Everett D, De Langhe S, Ortiz L A, Thorburn A, Riches DWH. Protein Tyrosine Phosphatase-N13 Promotes Myofibroblast Resistance to Apoptosis in Idiopathic Pulmonary Fibrosis. Am J Respir Crit Care Med 2018; 198: 914-927.
14. Redente E F, Keith R C, Janssen W, Henson P M, Ortiz L A, Downey G P, Bratton D L, Riches D W. Tumor necrosis factor-alpha accelerates the resolution of established pulmonary fibrosis in mice by targeting profibrotic lung macrophages. Am J Respir Cell Mol Biol 2014; 50: 825-837.
15. Darrah E, Giles J T, Ols M L, Bull H G, Andrade F, Rosen A. Erosive rheumatoid arthritis is associated with antibodies that activate PAD4 by increasing calcium sensitivity. Sci Transl Med 2013; 5: 186ra165.
16. Solomon J J, Matson S, Kelmenson L B, Chung J H, Hobbs S B, Rosas I O, Dellaripa P F, Doyle T J, Poli S, Esposito A J, Visser A, Marin A I, Amigues I, Fernandez Perez E R, Brown K K, Mahler M, Heinz D, Cool C, Deane K D, Swigris J J, Demoruelle M K. IgA Antibodies Directed Against Citrullinated Protein Antigens Are Elevated in Patients With Idiopathic Pulmonary Fibrosis. Chest 2020; 157: 1513-1521.
17. Li F J, Surolia R, Li H, Wang Z, Liu G, Kulkarni T, Massicano A V F, Mobley J A, Mondal S, de Andrade J A, Coonrod S A, Thompson P R, Wille K, Lapi S E, Athar M, Thannickal V J, Carter A B, Antony V B. Citrullinated vimentin mediates development and progression of lung fibrosis. Sci Transl Med 2021; 13.
18. Myasoedova E, Crowson C S, Turesson C, Gabriel S E, Matteson E L. Incidence of extraarticular rheumatoid arthritis in Olmsted County, Minnesota, in 1995-2007 versus 1985-1994: a population-based study. J Rheumatol 2011; 38: 983-989.
19. Olson A L, Swigris J J, Sprunger D B, Fischer A, Fernandez-Perez E R, Solomon J, Murphy J, Cohen M, Raghu G, Brown K K. Rheumatoid arthritis-interstitial lung disease-associated mortality. Am J Respir Crit Care Med 2011; 183: 372-378.
20. Swigris J J, Yorke J, Sprunger D B, Swearingen C, Pincus T, du Bois R M, Brown K K, Fischer A. Assessing dyspnea and its impact on patients with connective tissue disease-related interstitial lung disease. Respir Med 2010; 104: 1350-1355.
21. Tsuchiya Y, Takayanagi N, Sugiura H, Miyahara Y, Tokunaga D, Kawabata Y, Sugita Y. Lung diseases directly associated with rheumatoid arthritis and their relation to outcome. The European respiratory journal: official journal of the European Society for Clinical Respiratory Physiology 2010.
22. Flaherty K R, Wells A U, Cottin V, Devaraj A, Walsh S L F, Inoue Y, Richeldi L, Kolb M, Tetzlaff K, Stowasser S, Coeck C, Clerisme-Beaty E, Rosenstock B, Quaresma M, Haeufel T, Goeldner R G, Schlenker-Herceg R, Brown K K, Investigators I T. Nintedanib in Progressive Fibrosing Interstitial Lung Diseases. N Engl J Med 2019; 381: 1718-1727.
23. Chirivi R G S, van Rosmalen J W G, van der Linden M, Euler M, Schmets G, Bogatkevich G, Kambas K, Hahn J, Braster Q, Soehnlein O, Hoffmann M H, Es H, Raats J M H. Therapeutic ACPA inhibits NET formation: a potential therapy for neutrophil-mediated inflammatory diseases. Cell Mol Immunol 2020.
24. Linnemann R W, Yadav R, Zhang C, Sarr D, Rada B, Stecenko A A. Serum anti-PAD4 autoantibodies are present in cystic fibrosis children and increase with age and lung disease severity. Autoimmunity 2022; 55: 109-117.
25. Giles J T, Darrah E, Danoff S, Johnson C, Andrade F, Rosen A, Bathon J M.

Association of cross-reactive antibodies targeting peptidyl-arginine deiminase 3 and 4 with rheumatoid arthritis-associated interstitial lung disease. PLoS One 2014; 9: e98794.
26. Kelly C A, Saravanan V, Nisar M, Arthanari S, Woodhead F A, Price-Forbes A N, Dawson J, Sathi N, Ahmad Y, Koduri G, Young A, British Rheumatoid Interstitial Lung N. Rheumatoid arthritis-related interstitial lung disease: associations, prognostic factors and physiological and radiological characteristics—a large multicentre UK study. Rheumatology (Oxford) 2014; 53: 1676-1682.
27. Kim E J, Elicker B M, Maldonado F, Webb W R, Ryu J H, Van Uden J H, Lee J S, King T E, Jr., Collard H R. Usual interstitial pneumonia in rheumatoid arthritis-associated interstitial lung disease. Eur Respir J 2010; 35: 1322-1328.
28. Solomon J J, Chung J H, Cosgrove G P, Demoruelle M K, Fernandez-Perez E R, Fischer A, Frankel S K, Hobbs S B, Huie T J, Ketzer J, Mannina A, Olson A L, Russell G, Tsuchiya Y, Yunt Z X, Zelarney P T, Brown K K, Swigris J J. Predictors of mortality in rheumatoid arthritis-associated interstitial lung disease. Eur Respir J 2016; 47: 588-596.
29. Assayag D, Lubin M, Lee J S, King T E, Collard H R, Ryerson C J. Predictors of mortality in rheumatoid arthritis-related interstitial lung disease. Respirology 2014; 19: 493-500.
30. Humphries S M, Swigris J J, Brown K K, Strand M, Gong Q, Sundy J S, Raghu G, Schwarz M I, Flaherty K R, Sood R, O'Riordan T G, Lynch D A. Quantitative high-resolution computed tomography fibrosis score: performance characteristics in idiopathic pulmonary fibrosis. Eur Respir J 2018; 52.
31. Guiot J, Henket M, Corhay J L, Moermans C, Louis R. Sputum biomarkers in IPF: Evidence for raised gene expression and protein level of IGFBP-2. IL-8 and MMP-7. PLoS One 2017; 12: e0171344.
32. Njock M S, Guiot J, Henket M A, Nivelles O, Thiry M, Dequiedt F, Corhay J L, Louis R E, Struman I. Sputum exosomes: promising biomarkers for idiopathic pulmonary fibrosis. Thorax 2019; 74: 309-312.
33. Schupp J C, Khanal S, Gomez J L, Sauler M, Adams T S, Chupp G L, Yan X, Poli S, Zhao Y, Montgomery R R, Rosas I O, Dela Cruz C S, Bruscia E M, Egan M E, Kaminski N, Britto C J. Single Cell Transcriptional Archetypes of Airway Inflammation in Cystic Fibrosis. Am J Respir Crit Care Med 2020.
34. Lay J C, Peden D B, Alexis N E. Flow cytometry of sputum: assessing inflammation and immune response elements in the bronchial airways. Inhal Toxicol 2011; 23: 392-406.
35. Harris M L, Darrah E, Lam G K, Bartlett S J, Giles J T, Grant A V, Gao P, Scott W W, Jr., El-Gabalawy H, Casciola-Rosen L, Barnes K C, Bathon J M, Rosen A. Association of autoimmunity to peptidyl arginine deiminase type 4 with genotype and disease severity in rheumatoid arthritis. Arthritis Rheum 2008; 58: 1958-1967.
36. Halvorsen E H, Pollmann S, Gilboe I M, van der Heijde D, Landewe R, Odegard S, Kvien T K, Molberg O. Serum IgG antibodies to peptidylarginine deiminase 4 in rheumatoid arthritis and associations with disease severity. Ann Rheum Dis 2008; 67: 414-417.
37. Zhao J, Zhao Y, He J, Jia R, Li Z. Prevalence and significance of anti-peptidylarginine deiminase 4 antibodies in rheumatoid arthritis. J Rheumatol 2008; 35: 969-974.
38. Demoruelle M K, Wang H, Davis R L, Visser A, Hoang J, Norris J M, Holers V M, Deane K D, Darrah E. Anti-peptidylarginine deiminase-4 antibodies at mucosal sites can activate peptidylarginine deiminase-4 enzyme activity in rheumatoid arthritis. Arthritis Res Ther 2021; 23: 163.
39. Auger I, Martin M, Balandraud N, Roudier J. Rheumatoid arthritis-specific autoantibodies to peptidyl arginine deiminase type 4 inhibit citrullination of fibrinogen. Arthritis Rheum 2010; 62: 126-131.
40. Zhou Y, Chen B, Mittereder N, Chaerkady R, Strain M, An L L, Rahman S, Ma W, Low C P, Chan D, Neal F, Bingham C O, 3rd, Sampson K, Darrah E, Siegel R M, Hasni S, Andrade F, Vousden K A, Mustelin T, Sims G P. Spontaneous Secretion of the Citrullination Enzyme PAD2 and Cell Surface Exposure of PAD4 by Neutrophils. Font Immunol 2017; 8: 1200.
41. Shi J, Darrah E, Sims G P, Mustelin T, Sampson K, Konig M F, Bingham C O, 3rd, Rosen A, Andrade F. Affinity maturation shapes the function of agonistic antibodies to peptidylarginine deiminase type 4 in rheumatoid arthritis. Ann Rheum Dis 2018; 77: 141-148.
42. Ortiz L A, Lasky J, Hamilton R F, Jr., Holian A, Hoyle G W, Banks W, Peschon J J, Brody A R, Lungarella G, Friedman M. Expression of TNF and the necessity of TNF receptors in bleomycin-induced lung injury in mice. Exp Lung Res 1998; 24: 721-743.
43. Martinez J A, King T E, Jr., Brown K, Jennings C A, Borish L, Mortenson R L, Khan T Z, Bost T W, Riches D W. Increased expression of the interleukin-10 gene by alveolar macrophages in interstitial lung disease. Am J Physiol 1997; 273: L676-683.
44. Frankel S K, Cosgrove G P, Cha S I, Cool C D, Wynes M W, Edelman B L, Brown K K, Riches D W. TNF-alpha sensitizes normal and fibrotic human lung fibroblasts to Fas-induced apoptosis. Am J Respir Cell Mol Biol 2006; 34: 293-304.

What is claimed is:

1. A monoclonal antibody comprising:
a. a heavy variable chain comprising SEQ ID NO:1 and a light variable chain comprising SEQ ID NO:2;
b. a heavy variable chain comprising SEQ ID NO:3 and a light variable chain comprising SEQ ID NO:4;
c. a heavy variable chain comprising SEQ ID NO:5 and a light variable chain comprising SEQ ID NO:6;
d. a heavy variable chain comprising SEQ ID NO:7 and a light variable chain comprising SEQ ID NO:8; or
e. a heavy variable chain comprising SEQ ID NO:9 and a light variable chain comprising SEQ ID NO:10.

2. The monoclonal antibody of claim 1, wherein the antibody is a human monoclonal antibody.

3. A composition comprising at least one monoclonal antibody of claim 1.

4. The composition of claim 3, wherein the composition further comprises an excipient or at least one pharmaceutically acceptable carrier.

5. A kit comprising the composition of claim 3 or claim 4.

6. A method of treating pulmonary fibrosis in a subject in need of treatment thereof, the method comprising administering to the subject a therapeutically effective amount of a composition of claim 3 or claim 4.

7. The method of claim 6, wherein the subject is suffering from idiopathic lung fibrosis.

8. The method of claim 6, wherein the subject is suffering from interstitial lung disease.

9. The method of any of claims 6-8, wherein the subject is suffering from rheumatoid arthritis.
